(19) United States
(12) Patent Application Publication
(10) Pub. No.: US 2022/0145303 A1
(43) Pub. Date: May 12, 2022

(54) FRAGILE X MENTAL RETARDATION PROTEIN INTERFERING OLIGONUCLEOTIDES AND METHODS OF USING SAME

(71) Applicant: Nogra Pharma Limited, Dublin 2 (IE)

(72) Inventors: Francesca Viti, Salorino (CH); Salvatore Bellinvia, Mendrisio (CH)

(21) Appl. No.: 17/433,528

(22) PCT Filed: Feb. 26, 2020
(86) PCT No.: PCT/EP2020/055071

§ 371 Date: Aug. 24, 2021

Related U.S. Application Data
(60) Provisional application No. 62/810,697, filed on Feb. 26, 2019.

(51) Int. Cl. C12N 15/113; A61P 1/00; A61P 29/00; A61P 35/00

(52) U.S. Cl. CPC C12N 15/1135; A61P 1/00; A61P 29/00; A61P 35/00; C12N 2310/11; C12N 2310/3341; C12N 2310/3125; C12N 2310/14; C12N 2310/315

(57) ABSTRACT

Disclosed herein are antisense oligonucleotide sequences against fragile X mental retardation protein (FMRP) and methods of using the same for treating bowel diseases such as colorectal cancer and inflammatory bowel disease (e.g., Crohn's disease and ulcerative colitis), associated with elevated activity or expression of FMRP. Also disclosed are pharmaceutical compositions containing an FMRP antisense oligonucleotide useful for treating a bowel disease and manufacture of medicaments containing a disclosed FMRP antisense oligonucleotide to be used in treating a bowel disease.

### CROSS-REFERENCE TO RELATED APPLICATIONS

This application claims the benefit of and priority to U.S. Provisional Patent Application No. 62/810,697, filed Feb. 26, 2019, the disclosure of which is hereby incorporated by reference herein in its entirety for all purposes.

### BACKGROUND

RNA-binding proteins (RBPs) are involved in virtually all steps of the post-transcriptional regulatory process, dictating the fate and function of each transcript in the cell and ensuring cellular homeostasis. RBPs establish highly dynamic interactions with proteins as well as with coding and non-coding RNAs, creating functional units called ribonucleoprotein complexes that regulate RNA splicing, polyadenylation, stability, localization, translation, and degradation.

It has now become clear that RBPs are dysregulated in different types of cancers, influencing the expression and function of pro-tumorigenic and tumor-suppressor proteins, and inflammatory mediators. Several studies have provided evidence that RBPs are abnormally expressed in cancer relative to adjacent normal tissues, and their expression correlates with patients' prognosis. In recent years, fragile X mental retardation protein (FMRP) is gaining recognition for being of pivotal importance in controlling the development and growth of many different types of human cancer. Mutations or absence of FMRP cause fragile X syndrome (FXS), the most frequent form of inherited intellectual disability in humans. FMRP is an RBP involved in multiple steps of RNA metabolism. In the brain, its functional absence causes impaired synaptic plasticity due to defects in cytoskeletal organization and receptor mobility at synapses. Depending on the identity of the target mRNA, the presence of non-coding RNA and/or the cellular context, FMRP can act as a negative regulator of translation, modulate the stability of mRNA, regulate mRNA transport, or affect RNA editing. Of note, FMRP-regulated mRNA are involved in several mechanisms controlling cancer progression and metastasis.

Converging evidence highlights the involvement of FMRP in different types of cancer: the FMR1 gene, which encodes FMRP, is expressed in different tissues and cancer cell types; the FMR1 autosomal paralog and interactor, FXR1, was recently identified as a predictor of distant metastasis in triple-negative breast cancer; and several FMRP mRNA targets are involved in cancer progression. Moreover, compared with rates in the general population, the standardized incidence of cancer in FSX patients is significantly lower and FSX patients may be protected against certain forms of cancer.

Colorectal cancer (CRC) is one of the most common cancers worldwide, causing over a half-million deaths every year. The model of CRC tumorigenesis includes several genetic changes that are required for cancer initiation and progression. These changes are largely dictated by alterations of oncogenic and/or tumor-suppressive signaling pathways which are responsible for the progression from normal mucosa to an adenomatous polyp and then to carcinoma. It has become clear that colon tumor cells highjack post-transcriptional mechanisms that enable swift and robust adjustment of protein expression levels in response to intrinsic and extracellular signals, leading to cellular adaptation to the local microenvironment.

Epidemiological/genetic studies have also documented frequent development of immune-related disorders (e.g., thyroiditis, rheumatoid arthritis, Sjögren's syndrome, systemic lupus erythematosus, and multiple sclerosis) in women carrying pre-mutation alleles of FMR1 comprising an expanded trinucleotide (CGG) repeat element. Although it remains unknown the basic mechanism by which such FMRP gene alterations cause predisposition to immune pathologies, two recent studies documented an altered cytokine profile in children with FSX.

Inflammatory bowel disease (IBD) is a chronic inflammatory disorder of the gastrointestinal tract suffered by approximately 1.4 million patients in the United States. It is one of the five most prevalent gastrointestinal disease burdens in the United States, with an overall health care cost of more than $1.7 billion. Each year in the United States, IBD accounts for more than 700,000 physician visits, 100,000 hospitalizations, and disability in 119,000 patients. No medical cure currently exists, so disease management requires a lifetime of care.

The two most common forms of IBD are Crohn's disease and ulcerative colitis. Although Crohn's disease can affect the entire gastrointestinal tract, it primarily affects the ileum (the distal or lower portion of the small intestine) and the large intestine. Ulcerative colitis primarily affects the colon and the rectum. The etiology of inflammatory bowel disease is not completely understood, although both environmental and genetic factors are believed to play a role in the disease. Environmental components may include alterations in flora of the gut which are affected by exposure to ingested foods and medications.

IBD is associated with abdominal pain, vomiting, diarrhea, rectal bleeding, severe cramps, muscle spasms, weight loss, malnutrition, fever, and anemia. Patients with IBD may also suffer from skin lesions, joint pain, eye inflammation, and liver disorders, and children suffering from ulcerative colitis may suffer from growth defects. Although rarely fatal, these symptoms decrease quality of life for patients.

Thus, there is a pressing need to develop reliable methods of treating bowel diseases such as CRC and IBD. There is a further need to identify methods of treatment that provide effective and permanent relief from symptoms across a broad spectrum of patients and which are not associated with negative side effects or cycles of remission and/or inflammation.

### SUMMARY

Described herein are antisense oligonucleotides that inhibit the expression of fragile X mental retardation protein (FMRP), and methods of using the same. In some embodiments, the present disclosure provides a method of treating a bowel disease in a patient in need thereof comprising administering to the patient an effective amount of an antisense oligonucleotide that inhibits the expression of FMRP. In some embodiments, the bowel disease may be colorectal cancer or an inflammatory bowel disease such as Crohn's disease or ulcerative colitis. In some embodiments, the antisense oligonucleotide induces necroptosis.

In some embodiments, the present disclosure provides a method of treating a solid tumor, tumor invasion, or tumor metastasis in a patient in need thereof comprising administering to the patient an effective amount of an antisense oligonucleotide that inhibits the expression of FMRP. In some embodiments, the present disclosure provides a method of preventing or ameliorating tumor invasion or tumor metastasis. In certain embodiments, the present disclosure provides a method of preventing or ameliorating colorectal cancer tumor invasion or colorectal cancer tumor metastasis.

In some embodiments, the antisense oligonucleotide that inhibits the expression of FMRP comprises a sequence selected from the group consisting of:

(SEQ ID NO: 1)
5′-CCACCACCAGCTCCTCCA-3′,
(SEQ ID NO: 2)
5′-CTTCCACCACCAGCTCCT-3′,
(SEQ ID NO: 3)
5′-TCCACCACCAGCTCCTCC-3′,
(SEQ ID NO: 4)
5′-CTTCCACCACCAGCTCC-3′,
and
(SEQ ID NO: 5)
5′-TCACCCTTTATCATCCTC-3′,

or a complement thereof.

In further embodiments, the antisense oligonucleotide that inhibits the expression of FMRP comprises a sequence selected from the group consisting of:

(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,

or a complement thereof.

In some embodiments, the antisense oligonucleotide that inhibits the expression of FMRP consists of a sequence selected from the group consisting of:

(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,

or a complement thereof.

In some embodiments, the antisense oligonucleotide may be an antisense oligonucleotide wherein at least one internucleoside linkage of the sequence is a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, or a boranophosphate linkage. In a particular embodiment, at least one internucleoside linkage of the antisense oligonucleotide sequence is a phosphorothioate linkage. In some embodiments, all of the internucleoside linkages of the antisense oligonucleotide sequence are phosphorothioate linkages. In some embodiments, at least one nucleoside linkage of the sequence is a methylphosphonate linkage.

The number of nucleotides included in FMRP antisense oligonucleotides described herein may vary. For example, in some embodiments, the antisense oligonucleotide is from 20 to 40 nucleotides in length. In some embodiments, the antisense oligonucleotide is from 20 to 24 nucleotides in length.

In some embodiments, the FMRP antisense oligonucleotide induces necroptosis via activation of the receptor-interacting protein kinase 1 (RIP1 or RIPK1)—receptor-interacting protein kinase 3 (RIP3 or RIPK3)—mixed lineage kinase domain-like protein (MLKL) complex. For example, the FMRP antisense oligonucleotide may increase the expression of RIPK1.

In some embodiments, the FMRP antisense oligonucleotide comprises one or more ribonucleotides, one or more deoxyribonucleotides, or a mixture of ribonucleotides and deoxyribonucleotides.

In some embodiments, the FMRP antisense oligonucleotide comprises one or more modified nucleoside, for example, 5-methyl cytidine, 5-methyl-2′-deoxycytidine, deoxycytidine, 5-methyl-2′-deoxycytidine 5′-monophosphate, or 5-methyl-2′-deoxycytidine-5′-monophosphorothioate. In certain embodiments, the FMRP antisense oligonucleotide comprises one or more modified nucleoside, for example, 2′-O-methylcytidine, 2′-O-methylguanosine, 2′-O-methylthymidine, 2′-O-methyluridine, or 2′-O-methyladenosine. In some embodiments, the FMRP antisense oligonucleotide comprises one or more modified nucleotide, for example, 5-methyl cytosine or 5-methylguanine. In some embodiments, the FMRP antisense oligonucleotides comprises one or more modified nucleotide, for example, 2′-O-(2-methoxyethyl) nucleosides, 2′-deoxy-2′-fluoro nucleosides, or 2′-fluoro-O-D-arabinonucleosides.

In some embodiments, the FMRP antisense oligonucleotide comprises bridged nucleic acids, locked nucleic acids (LNA), constrained ethyl (cET) nucleic acids, tricyclo-DNAs (tcDNA), 2′-O,4′-C-ethylene linked nucleic acids (ENA), or peptide nucleic acids (PNA).

In some embodiments, the FMRP antisense oligonucleotide is an FMRP siRNA, or a pharmaceutically acceptable salt thereof.

In some embodiments, the FMRP antisense oligonucleotide is administered to the patient enterally or pareneterally. For example, in some embodiments, the FMRP antisense oligonucleotide is administered to the patient orally, sublingually, gastrically, or rectally. In other embodiments, administration of the FMRP antisense oligonucleotide to the patient is intravenous, intratumoral, intrajejunal, intraileal, intracolonic, or intrarectal.

In particular embodiments, the FRMP antisense oligonucleotide of the present disclosure is for the treatment of a bowel disease in a human patient.

Also described herein is a pharmaceutically acceptable composition comprising an FMRP antisense oligonucleotide described herein and a pharmaceutically acceptable carrier. In some embodiments, the pharmaceutical composition is suitable for oral, sublingual, gastric, or rectal administration. In other embodiments the pharmaceutical composition is suitable for intravenous, intratumoral, intrajejunal, intraileal, intracolonic, or intrarectal administration.

Also described herein is use of an FMRP antisense oligonucleotide in the manufacture of a medicament for the treatment of a bowel disease. In some embodiments, the bowel disease is CRC, or an IBD such as Crohn's disease or ulcerative colitis. In other embodiments, the medicament is administered to the patient enterally or parenterally. For example, in some embodiments, the medicament is suitable for oral, sublingual, gastric, or rectal administration. In some embodiments, the medicament is suitable for intravenous, intratumoral, intrajejunal, intraileal, intracolonic, or intrarectal administration. In some embodiments, the medicament is for treatment of a bowel disease in a human.

In some embodiments, the present disclosure provides a use of an FMRP antisense oligonucleotide in the manufacture of a medicament for the treatment of a solid tumor, tumor invasion, or tumor metastasis.

### BRIEF DESCRIPTION OF THE DRAWINGS

FIG. 1A are immunohistological images showing staining for FMRP in human CRC samples (IgG=Isotype control; NC=colon sample of a normal control; P=peritumor sample; T=tumor sample). FIG. 1B is a line graph showing FMRP mRNA expression levels in peritumor (P) and CRC tumor samples (T) as determined by RT-PCR analysis (**p<0.01). FIG. 1C is a Western blot showing FMRP protein expression in paired peritumor (P) and CRC tumor samples (T) from CRC patients. FIG. 1D is a graph showing FMRP protein expression relative to β-actin expression in paired peritumor (P) and CRC tumor samples (T) as measured by quantitative densitometry of Western blot analyses (relative expression shown as arbitrary units (a.u.)).

FIG. 2A are endoscopic images of colons of untreated wild-type (WT) and FMR1 knock-out (KO) mice, or mice 21 weeks following intraperitoneal injection of Azoxymethane (AOM). FIG. 2B are bar graphs showing tumor load (left) and tumor size (right) in the colons of WT and FMR1 KO mice 21 weeks following intraperitoneal injection of AOM (*p<0.05, **p<0.01). FIG. 2C is a graph showing the % survival of WT and FMR1 KO mice intraperitoneally injected with AOM. FIG. 2D are histological images showing hematoxylin and eosin (H&E) staining of samples prepared from AOM-induced tumors in WT and FMR1 KO mice. FIG. 2E is a Western blot showing FMRP and β-actin (loading control) protein expression in colon samples prepared from untreated WT mice, and AOM-treated mice (P=peritumor sample; T=CRC tumor sample). FIG. 2F are histological images showing TUNEL staining of samples prepared from AOM-induced tumors in WT and FMR1 KO mice. FIG. 2G are immunohistological images showing staining for Ki67 in AOM-induced tumor samples from WT and FMR1 KO mice.

FIG. 3A is a representative Western blot showing FMRP and β-actin (loading control) protein expression in DLD-1 and HCT-116 human colon cancer cell lines, and the HCEC-1ct non-cancerous human colonic epithelial cell line. FIG. 3B is a graph showing FMRP protein expression relative to β-actin expression in DLD-1, HCT-116, and HCEC-1ct cell lines as determined by densitometry analysis of the Western blots described in FIG. 3A (relative expression shown as arbitrary units (a.u.)) (**p<0.01, ***p<0.001). FIG. 3C are immunofluorescence images showing DLD-1, HCT-116, and HCEC-1ct cells stained for FMRP expression. FIGS. 3D, 3G, and 3I are Western blots showing FMRP and β-actin (loading control) protein expression in DLD-1 cells, HCT-116 cells, and HCEC-1ct cells, respectively, treated with sense or antisense FMRP oligonucleotides (U=untreated; S=sense; AS=antisense) for 48 hours. FIGS. 3E, 3H, and 3J show flow cytometry dot plots of DLD-1 cells, HCT-116 cells, and HCEC-1ct cells, respectively, treated with sense or antisense FMRP oligonucleotides (Neg=negative staining control; U=untreated; S=sense; AS=antisense) and stained with propidium iodide (PI) and Annexin V (AnnV). FIGS. 3F and 3K show bar graphs of the corresponding flow cytometry dot plot data of FIGS. 3E and 3J, respectively (**p<0.01).

FIG. 4A are flow cytometry histograms showing staining for caspase 8 and caspase 3 in untreated CRC cells (U), CRC cells treated with sense FMRP oligonucleotide (S), CRC cells treated with antisense FMRP oligonucleotide (AS), or CRC cell treated with staurosporin (Stauro). FIG. 4B is a bar graph of the corresponding flow cytometry data of FIG. 4A (***p<0.001). FIG. 4C is a bar graph showing relative cell death as determined by flow cytometry analysis of PI and AnnV-stained CRC cells. CRC cells received no pre-treatment or were pre-treated with pan-caspase inhibitor (Cas in) prior to treatment with sense FMRP oligonucleotide (S), antisense FMRP oligonucleotide (AS), or staurosporin (Stauro). FIG. 4D are flow cytometry dot plots showing Brdu and PI staining of untreated DLD-1 cells (U) or DLD-1 cells treated with sense FMRP oligonucleotide (S) or antisense FMRP oligonucleotide (AS). FIG. 4E is a bar graph showing the corresponding flow cytometry dot plot data of FIG. 4D. FIG. 4F is a bar graph showing HCT-116 cell proliferation assessed by BrdU incorporation assay (U=untreated; S=sense; AS=antisense) for 36 hours. FIG. 4G is a bar graph showing cell cycle distribution in HCT-116 cells treated with sense or antisense FMRP oligonucleotides (U=untreated; S=sense; AS=antisense) for 36 hours.

FIG. 5 shows an RNA immunoprecipitation of human CRC cell lysates. FIG. 5A is a Western blot confirming specificity of the FMRP antibody used for RNA immunoprecipitation of human CRC cell lysates. FIG. 5B is a bar graph showing enrichment of FMRP co-precipitated mRNA as determined by RT-PCR analysis using primers specific for actin, E-cadherin, RIPK3, and RIPK1. FIG. 5C is a bar graph showing enrichment of FMRP co-precipitated mRNA from CRC cell line lysates as determined by RT-PCR analysis using primers specific for actin, vimentin, RIPK3, and RIPK1.

FIG. 6A is a representative Western blot showing protein/phospho-protein expression of FMRP, phospho-RIPK1 (pRIPK1), RIPK1, phospho-RIPK3 (pRIPK3), RIPK3, phospo-MLKL (pMLKL), MLKL, and β-actin (loading control) in untreated human colon cell line (U), or human colon cell line treated with FMRP sense oligonucleotide (S), or human colon cell line treated with FMRP antisense oligonucleotide (AS). FIGS. 6B, 6C, and 6D are bar graphs showing pRIPK1, pRIPK3, and pMLKL expression levels, respectively, as determined by densitometry analysis of the Western blots described in FIG. 6A (**p<0.01, ***p<0.001, ****p<0.0001). FIGS. 6E and 6F are bar graphs showing relative cell death as determined by flow cytometry analysis of PI and AnnV-stained human CRC cell lines treated with RIPK1-specific inhibitor (NEC1) or MLKL-specific inhibitor (NSA), respectively, and either sense FMRP oligonucleotide (S) or anti-sense FMRP oligonucleotide (AS). FIG. 6G is a representative Western blot showing protein/phospho-protein expression of FMRP, pRIPK1, RIPK1, pRIPK3, RIPK3, pMLKL, MLKL, and β-actin (loading control) in untreated HCEC-1ct cells, HCEC-1ct cells treated with FMRP sense oligonucleotide (S), or HCEC-1ct cells treated with FMRP antisense oligonucleotides (AS).

FIG. 7A is a line graph showing RT-PCR analysis of CREB mRNA expression levels in peritumor (P) and CRC tumor samples (T) (***p<0.001). FIG. 7B is a representative Western blot showing CREB, FMRP and β-actin (loading control) protein expression in match paired peritumor (P) and CRC tumor samples (T) from CRC patients. FIG. 7C is a bar graph showing CREB protein expression relative to β-actin expression as determined by densitometry analysis of the Western blots described in FIG. 7B (**p<0.01). FIG. 7D is a representative Western blot showing CREB, FMRP, and β-actin (loading control) protein expression of untreated CRC cell lines (U), CRC cell lines treated with FMRP sense oligonucleotide (S), or CRC cell lines treated with FMRP antisense oligonucleotides (AS). FIGS. 7E and 7F are bar graphs showing CREB and FMRP protein expression, respectively, relative to β-actin expression as determined by densitometry analysis of the Western blot described in FIG. 7D (*p<0.05, **p<0.01, ***p<0.001).

FIG. 8A are microscopic images showing cell migration of HCT-116 cells treated with sense or antisense FMRP oligonucleotides (U=untreated; S=sense; AS=antisense) and seeded onto each side of an Ibidi® culture insert at the time of insert removal (TO), and after 24 hours and 48 hours incubation (T24 and T48, respectively). FIG. 8B is a bar graph showing the percentage of cell-covered area corresponding to the microscopic images of FIG. 8A (T24: U vs AS, **p<0.01; S vs AS *p<0.05; T48: U vs AS and S vs AS, ***p<0.001). FIGS. 8C and 8D are microscopic images and a corresponding cell-count bar graph, respectively, of HCT-116 cells, treated with sense or antisense FMRP oligonucleotides (U=untreated; S=sense; AS=antisense), that migrated across Matrigel-coated Transwell® inserts after 48 hours incubation (***p<0.001, ****p<0.0001). FIG. 8E is a representative Western blot showing FMRP, E-cadherin, β-catenin, and β-actin (loading control) protein expression in HCT-116 cells treated with sense or antisense FMRP oligonucleotides (U=untreated; S=sense; AS=antisense) for 48 hours. FIGS. 8F and 8G are bar graphs showing E-cadherin and β-catenin protein expression, respectively, relative to β-actin expression in HCT-116 cells as determined by densitometry analysis of the Western blots described in FIG. 8E (**p<0.01, ***p<0.001).

FIG. 9A is a representative Western blot showing FMRP, MCC, and β-actin (loading control) protein expression in untreated HCT-116 cells (U) or HCT-116 cells treated with sense oligonucleotide (S), or antisense FMRP oligonucleotide (AS) for 48 hours. FIG. 9B is a bar graph showing MCC protein expression relative to β-actin expression in HCT-116 cells as determined by densitometry analysis of the Western blot described in FIG. 9A. FIG. 9C is a representative Western blot showing MCC, FMRP, E-cadherin, β-catenin, and β-actin (loading control) protein expression in untreated HCT-116 cells (U) or HCT-116 cells treated with sense oligonucleotide (S), antisense FMRP oligonucleotide (AS), and/or control siRNA (siRNA Ctrl) or siRNA specific for MCC (siRNA MCC) for 48 hours. FIGS. 9D and 9E are bar graphs showing E-cadherin and β-catenin protein expression, respectively, relative to β-actin expression in HCT-116 cells as determined by densitometry analysis of the Western blots described in FIG. 9C (*p<0.05, **p<0.01). FIG. 9F are microscopic images showing cell migration of HCT-116 cells treated with sense or antisense FMRP oligonucleotides (U=untreated; S=sense; AS=antisense), and/or siRNA Ctrl or siRNA MCC and seeded onto each side of an Ibidi® culture insert at the time of insert removal (TO), and after 24 hours and 48 hours incubation (T24 and T48, respectively). FIG. 9G is a bar graph showing the percentage of cell-covered area corresponding to the microscopic images of FIG. 9F (T24: HCT-116 cells transfected with antisense FMRP oligonucleotide and siRNA MCC vs HCT-116 cells transfected with antisense FMRP oligonucleotide alone, ***p<0.001; T48: HCT-116 cells transfected with antisense FMRP oligonucleotide and siRNA MCC vs HCT-116 cells transfected with antisense FMRP oligonucleotide alone, ****p<0.0001).

### DETAILED DESCRIPTION

### FMRP Antisense Oligonucleotides

“Antisense oligonucleotide,” as used herein, refers to a short synthetic oligonucleotide sequence complementary to a messenger RNA (mRNA), which encodes for a target protein (e.g., FMRP). Antisense oligonucleotide sequences hybridize to the mRNA producing a double-stranded molecule that can lead to the activation of nucleases, which recognize and degrade the double-stranded molecule, thus preventing translation of the mRNA. Antisense oligonucleotides may include single-stranded DNA oligonucleotides, small hairpin RNAs (shRNAs), small interfering RNAs (siRNAs), and modified antisense oligonucleotides that include, but are not limited to, 2′-O-alkyl, peptide nucleic acid (PNA), locked nucleic acid (LNA), and morpholino oligomer chemistries.

In some embodiments, the antisense oligonucleotide can be a single-stranded nucleic acid molecule comprising nucleotide sequence that is complementary to a target mRNA (e.g., FMRP). For example, the antisense oligonucleotide can be a single-stranded DNA oligonucleotide having sequence complementary to a FMRP mRNA. Hybridization of the FRMP antisense oligonucleotide to the target mRNA produces a double-stranded DNA/RNA hybrid that can lead to the activation of ubiquitous nucleases, such as RNase H, which recognize and degrade DNA/RNA hybrid strands, thus preventing translation of the target protein (e.g., FMRP).

Alternatively, the antisense oligonucleotide can be double-stranded. The double-stranded antisense oligonucleotide can be comprised of a single oligonucleotide having self-complementary sense and anti-sense regions. In other embodiments, the double-stranded antisense oligonucleotide can be comprised of two separate oligonucleotides, wherein one oligonucleotide is a sense strand, and the other oligonucleotide is an antisense strand, and wherein the antisense strand has a nucleotide sequence that is complementary to a target mRNA (e.g., FMRP).

Antisense oligonucleotides can be designed such that the targeting portion of the incorporated nucleotide sequence of each antisense oligonucleotide is completely or almost completely complementary to the FMRP mRNA sequence. Incorporation of such complementary or nearly complementary nucleotide sequences allows one to engineer antisense oligonucleotides with a high degree of specificity for a given target. Specificity can be assessed via measurement of parameters such as dissociation constant, or other criteria such as changes in protein or RNA expression levels or other assays that measure FMRP activity or expression.

The present disclosure provides methods that include administration to the patient of a FMRP antisense oligonucleotide capable of targeting FMRP mRNA for degradation, interfering with mRNA splicing, or preventing FMRP gene expression or protein translation. The FMRP antisense oligonucleotides of the present disclosure can target various regions of the human FMRP mRNA for binding. The human FMRP mRNA has the sequence of NCBI Reference Sequences: NM_001185075 (SEQ ID NO: 18), NM_001185076 (SEQ ID NO: 19), NM_001185081 (SEQ ID NO: 20), NM_001185082 (SEQ ID NO: 21), or NM_002024 (SEQ ID NO: 22).

An FMRP antisense oligonucleotide, such as disclosed herein, may be an oligonucleotide sequence of 5 to 100 nucleotides in length, for example, 10 to 40 nucleotides in length, for example, 14 to 40 nucleotides in length, for example, 10 to 30 nucleotides in length, for example, 14 to 30 nucleotides in length, for example, 14 to 25 nucleotides in length, for example, 15 to 22 oligonucleotides in length, for example, 18 to 40 nucleotides in length, for example, 18 to 24 nucleotides in length, for example 20 to 40 nucleotides in length, or for example, 20 to 24 nucleotides in length. In some embodiments, an FMRP antisense oligonucleotide can be, for example, 20, 21, 22, 23, 24, 25, 26, 27, 28, 29, 30, 31, 32, 33, 34, 35, 36, 37, 38, 39 or 40 nucleotides in length. An FMRP antisense oligonucleotide may comprise an oligonucleotide sequence complementary to one or more than one portion of the FMRP mRNA sequence.

In some embodiments, FMRP antisense oligonucleotides of the disclosure can be, but are not limited to, small hairpin RNAs (shRNAs), small interfering RNAs (siRNAs), morpholino oligomers, microRNAs, and compositions that include such compounds, for example, compositions that include a pharmaceutically acceptable excipient.

In some embodiments of the disclosure, an antisense oligonucleotide targeting FMRP comprises a sequence or a portion of a sequence (e.g., comprises a sequence having 90%, 95%, or 99% identity over the length) selected from any one of:

(SEQ ID NO: 1)
5′-CCACCACCAGCTCCTCCA-3′,
(SEQ ID NO: 2)
5′-CTTCCACCACCAGCTCCT-3′,
(SEQ ID NO: 3)
5′-TCCACCACCAGCTCCTCC-3′,
(SEQ ID NO: 4)
5′-CTTCCACCACCAGCTCC-3′,
and
(SEQ ID NO: 5)
5′-TCACCCTTTATCATCCTC-3′,

or a complement thereof.

In further embodiments, an antisense oligonucleotide targeting FMRP comprises a sequence or a portion of a sequence (e.g., comprises a sequence having 90%, 95%, or 99% identity over the length) selected from any one of:

(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,

or a complement thereof.

In some embodiments, the FMRP antisense oligonucleotide of the present disclosure comprises one or more ribonucleotides, deoxyribonucleotides, or a mixture of ribonucleotides and deoxyribonucleotides.

In some embodiments, the FMRP antisense oligonucleotide of the present disclosure comprises one or more modified nucleoside selected from the group consisting of 5-methylcytidine, 5-methyl-2′-deoxycytidine, deoxycytidine, 5-methyl-2′-deoxycytidine 5′-monophosphate, and 5-methyl-2′-deoxycytidine-5′-monophosphorothioate.

In certain embodiments, the FMRP antisense oligonucleotide of the present disclosure comprises one or more modified nucleoside selected from the group consisting of 2′-O-methyl cytidine, 2′-O-methylguanosine, 2′-O-methylthymidine, 2′-O-methyluridine, and 2′-O-methyl adenosine.

In some embodiments, the FMRP antisense oligonucleotide of the present disclosure comprises one or more modified nucleotide selected from the group consisting of 5-methyl cytosine and 5-methylguanine.

In some embodiments, the FMRP antisense oligonucleotide of the present disclosure comprises one or more modified nucleoside selected from 2′-O-(2-methoxyethyl) nucleosides, 2′-deoxy-2′-fluoro nucleosides, and 2′-fluoro-β-D-arabinonucleosides.

In certain embodiments, the FMRP antisense oligonucleotide of the present disclosure comprises one or more of the group selected from bridged nucleic acids, locked nucleic acids (LNA), constrained ethyl (cET) nucleic acids, tricyclo-DNAs (tcDNA), 2′-O,4′-C-ethylene linked nucleic acids (ENA), and peptide nucleic acids (PNA).

In some embodiments, at least one internucleoside linkage of a disclosed FMRP antisense oligonucleotide may have a modified linkage, such as a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, and an aminoalkylphosphoramidate linkage, a thiophosphoramidate linkage, a thionoalkylphosphonate linkage, a thionoalkylphosphotriester linkage, a thiophosphate linkage, a selenophosphate linkage, and/or a boranophosphate linkage. For example, in some embodiments, one, two or more, e.g., all internucleoside linkage(s) of a disclosed FMRP antisense oligonucleotide may be phosphorothioate linkages. In other embodiments, one, two or more, e.g., all internucleoside linkage(s) of a disclosed FMRP antisense oligonucleotide may be methylphosphonate linkages.

For example, in one embodiment, the FMRP antisense oligonucleotide is a phosphorothioate antisense oligonucleotide against FMRP, comprising the sequence of 5′-CCACCACCAGCTCCTCCA-3′ (SEQ ID NO: 1), where each internucleotide linkage of the antisense oligonucleotide is a phosphorothioate linkage. In some embodiments, the antisense oligonucleotide against FMRP is a phosphorothioate antisense oligonucleotide comprising the sequence 5′-CCACCACCAGCTCCTCCA-3′ (SEQ ID NO: 1), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-CTTCCACCACCAGCTCCT-3′ (SEQ ID NO: 2), where each internucleotide linkage of the antisense oligonucleotide is a phosphorothioate linkage. In some embodiments, the antisense oligonucleotide against FMRP is a phosphorothioate antisense oligonucleotide comprising the sequence 5′-CTTCCACCACCAGCTCCT-3′ (SEQ ID NO: 2), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-TCCACCACCAGCTCCTCC-3′ (SEQ ID NO: 3), where each internucleotide linkage of the antisense oligonucleotide is a phosphorothioate linkage. In some embodiments, the antisense oligonucleotide against FMRP is a phosphorothioate antisense oligonucleotide comprising the sequence 5′-TCCACCACCAGCTCCTCC-3′ (SEQ ID NO: 3), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-CTTCCACCACCAGCTCC-3′ (SEQ ID NO: 4), where each internucleotide linkage of the antisense oligonucleotide is a phosphorothioate linkage. In some embodiments, the antisense oligonucleotide against FMRP is a phosphorothioate antisense oligonucleotide comprising the sequence 5′-CTTCCACCACCAGCTCC-3′ (SEQ ID NO: 4), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-TCACCCTTTATCATCCTC-3′ (SEQ ID NO: 5), where each internucleotide linkage of the antisense oligonucleotide is a phosphorothioate linkage. In some embodiments, the antisense oligonucleotide against FMRP is a phosphorothioate antisense oligonucleotide comprising the sequence 5′-TCACCCTTTATCATCCTC-3′ (SEQ ID NO: 5), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-TCCACCACCAGCTCCTCCAT-3′ (SEQ ID NO: 6), where each internucleotide linkage of the antisense oligonucleotide is a phosphorothioate linkage. In some embodiments, the antisense oligonucleotide against FMRP is a phosphorothioate antisense oligonucleotide comprising the sequence 5′-TCCACCACCAGCTCCTCCAT-3′ (SEQ ID NO: 6), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-ACTTCCACCACCAGCTCCTC-3′ (SEQ ID NO: 7), where each internucleotide linkage of the antisense oligonucleotide is a phosphorothioate linkage. In some embodiments, the antisense oligonucleotide against FMRP is a phosphorothioate antisense oligonucleotide comprising the sequence 5′-ACTTCCACCACCAGCTCCTC-3′ (SEQ ID NO: 7), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-TTCCACCACCAGCTCCTCCA-3′ (SEQ ID NO: 8), where each internucleotide linkage of the antisense oligonucleotide is a phosphorothioate linkage. In some embodiments, the antisense oligonucleotide against FMRP is a phosphorothioate antisense oligonucleotide comprising the sequence 5′-TTCCACCACCAGCTCCTCCA-3′ (SEQ ID NO: 8), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-ACTTCCACCACCAGCTCCT-3′ (SEQ ID NO: 9), where each internucleotide linkage of the antisense oligonucleotide is a phosphorothioate linkage. In some embodiments, the antisense oligonucleotide against FMRP is a phosphorothioate antisense oligonucleotide comprising the sequence 5′-ACTTCCACCACCAGCTCCT-3′ (SEQ ID NO: 9), where one or more than one internucleoside linkage of the antisense oligonucleotide is a phosphorothioate linkage.

In another embodiment, the FMRP antisense oligonucleotide is a phosphorothioate antisense oligonucleotide against FMRP, comprising the sequence of 5′-CTCACCCTTTATCATCCTCA-3′ (SEQ ID NO: 10), where each internucleotide linkage of the antisense oligonucleotide is a phosphorothioate linkage. In some embodiments, the antisense oligonucleotide against FMRP is a phosphorothioate antisense oligonucleotide comprising the sequence 5′-CTCACCCTTTATCATCCTCA-3′ (SEQ ID NO: 10), where one or more than one internucleoside linkage of the antisense oligonucleotide is a phosphorothioate linkage.

In some embodiments, an FMRP antisense oligonucleotide is an siRNA that comprises the nucleotide sequence of any one of SEQ ID NOs: 1-10, or a pharmaceutically acceptable salt thereof. For example, an FMRP siRNA can comprise a sequence of any one of:

(SEQ ID NO: 1)
5′-CCACCACCAGCTCCTCCA-3′,
(SEQ ID NO: 2)
5′-CTTCCACCACCAGCTCCT-3′,
(SEQ ID NO: 3)
5′-TCCACCACCAGCTCCTCC-3′,
(SEQ ID NO: 4)
5′-CTTCCACCACCAGCTCC-3′,
(SEQ ID NO: 5)
5′-TCACCCTTTATCATCCTC-3′,
(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,

or a pharmaceutically acceptable salt of an FMRP siRNA comprising the sequence of any one of SEQ ID NOs: 1-10.

In some embodiments, an FMRP siRNA comprises at least one internucleoside linkage selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In certain embodiments, at least one internucleoside linkage of the FMRP siRNA is a phosphorothioate linkage. For example, in some embodiments, 1, 2, 3, 4, 5, 6, 7, 8, 9, 10, 11, 12, 13, 14, 15, 16, 17, 18, 19, 1-5, 1-10, 1-14, 1-15, 1-16, 1-19, 5-10, 5-14, 5-15, 5-19, 10-14, 10-15, or 10-19 internucleoside linkages of the FMRP siRNA are phosphorothioate linkages. In some embodiments, all of the internucleoside linkages of the FMRP siRNA are phorphorothioate linkages.

In various embodiments, a disclosed FMRP antisense oligonucleotide (e.g., an FMRP siRNA) may optionally have at least one modified nucleobase, e.g., 5-methylcytosine, and/or at least one methylphosphonate nucleotide in the sequence, which is placed, for example, either at only one of the 5′ or 3′ ends, or at both 5′ and 3′ ends, or along the oligonucleotide sequence.

FMRP antisense oligonucleotides (e.g., an FMRP siRNA) may optionally include at least one modified sugar. For example, the sugar moiety of at least one nucleotide constituting the oligonucleotide can be a ribose in which the 2′-OH group may be replaced by any one selected from the group consisting of OR, R, R′OR, SH, SR, NH₂, NR₂, N₃, CN, F, Cl, Br, and I (wherein R is an alkyl or aryl and R′ is an alkylene).

In some embodiments, certain disclosed nucleotides may be modified or have variations, for example, certain cytidines within a disclosed FMRP antisense oligonucleotide (e.g., an FMRP siRNA) may be, e.g., 5-methyl-2′-deoxycytidine, including, but not limited to, 5-methyl-2′-deoxycytidine 5′-monophosphate and 5-methyl-2′-deoxycytidine-5′-monophosphorothioate.

In certain embodiments, FMRP antisense oligonucleotides (e.g., an FMRP siRNA) can include chemically modified nucleosides, for example, 2′-O-methyl (2′-OMe) ribonucleosides, for example, 2′-O-methyl cytidine, 2′-O-methylguanosine, 2′-O-methylthymidine, 2′-O-methyluridine, and/or 2′-O-methyladenosine. FMRP antisense oligonucleotides (e.g., an FMRP siRNA) described herein can also include one or more chemically modified bases, including a 5-methyl pyrimidine, for example, 5-methylcytosine, and/or a 5 methylpurine, for example, 5-methylguanine. In some embodiments, FMRP antisense oligonucleotides (e.g., an FMRP siRNA) can include one or more 2′-O-(2-methoxyethyl) (2′-MOE) nucleosides, 2′-deoxy-2′-fluoro nucleosides, 2′-fluoro-β-D-arabinonucleosides, bridged nucleic acids, locked nucleic acids (LNA), constrained ethyl (cET) nucleic acids, tricyclo-DNAs (tcDNA), 2′-O,4′-C-ethylene linked nucleic acids (ENA), and/or peptide nucleic acids (PNA).

In some embodiments, at least one of the internucleotide linkages of a contemplated antisense oligonucleotide (e.g., an FMRP siRNA) is an O,O-linked phosphorothioate. For example, each of the internucleotide linkages of SEQ ID NOs: 1-10 may be an O,O-linked phosphorothioate. In some embodiments, compositions disclosed herein may include a pharmaceutically acceptable salt, e.g., a sodium salt of the antisense oligonucleotide of a disclosed sequence, that optionally may include 1 to 24 or more O,O-linked phosphorothioate internucleotide linkages. Contemplated salts of oligonucleotides include those that are fully neutralized, e.g., each phosphorothioate linkage is associated with an ion such as Nat Oligonucleotides may include naturally occurring nucleobases, sugars, and covalent internucleoside (backbone) linkages as wells as non-naturally occurring portions.

Also provided herein are isotopologues of disclosed antisense oligonucleotides, pharmaceutical compositions containing the same, and methods of using the same. For example, in some embodiments, provided herein are deuterated antisense oligonucleotides of SEQ ID NOs: 1-10, including a plurality of hydrogens (H), wherein one or more hydrogens of the plurality of hydrogens are replaced by deuterium (D).

### Bowel Diseases

The present disclosure provides oligonucleotides for treatment and/or prevention of bowel diseases. As used herein, “bowel disease”, refers to any disease, disorder, and/or syndrome affecting the portion of the alimentary canal after the stomach, i.e. the small intestine, large intestine, colon, and rectum. For example, bowel diseases can include, but are not limited to, colon cancer, inflammatory bowel disease, familial adenomatous polyposis, Gardner syndrome, Turcot syndrome, Lynch syndrome, coeliac disease, gastrointestinal carcinoid tumors, small intestine cancer, duodenal cancer, small bowel cancer, and gastrointestinal stromal tumors. For example, provided herein are methods of treating a patient suffering from a bowel disease comprising administering to the patient an effective amount of a disclosed antisense oligonucleotide.

### Colorectal Cancer

“Colorectal cancer,” as used herein, refers to any cancer affecting the colon and/or rectum. Colorectal cancers can be caused by any one or more than one environmental and genetic factor that causes the progressive accumulation of genetic and/or epigenetic alterations that attenuate tumor suppressor genes and activate oncogenes in colonic or rectal epithelial cells. Colorectal neoplasms are often associated with a loss of genomic and/or epigenomic stability, which accelerates malignant transformation.

Significant heterogeneity exists in the specific gene mutations present in colorectal cancer and include, but are not limited to, alterations in APC, CTNNB1, KRAS, BRAF, SMAD4, TGFBR2, TP53, PIK3CA, ARID1A, SOX9, FAM123B, and ERBB2. Colorectal cancer is often initiated by mutations resulting in dysregulated Wnt signaling, and tumors progress upon further dysregulation of other signaling pathways including the RAS-RAF-MAPK, TGFβ, and PI3K-AKT pathways. Disclosed sequences may post-transcriptionally regulate the expression of oncogenes, tumor suppressors, and key signaling proteins in all of the Wnt, RAS-RAF-MAPK, TGFβ, and PI3K-AKT pathways. Provided herein are methods of treating patients suffering from colorectal cancer comprising administering to patients a disclosed antisense oligonucleotide.

### Inflammatory Bowel Disease

“Inflammatory bowel disease,” as used herein, refers to a number of chronic inflammatory diseases including Crohn's disease, ulcerative colitis, gastroduodenal Crohn's disease, Crohn's (granulomatous) colitis, collagenous colitis, lymphocytic colitis, ischaemic colitis, diversion colitis, Behcet's disease, microscopic colitis, ulcerative proctitis, proctosigmoiditis, jejunoileitis, left-sided colitis, pancolitis, ileocolitis, ileitis, and indeterminate colitis. Crohn's disease and ulcerative colitis are the two most common forms of inflammatory bowel disease. Inflammatory bowel disease is an autoimmune disease of the digestive system. Crohn's disease may be localized to any portion of the gastrointestinal tract, including the terminal ileum, and may impact all cell types of the gastrointestinal tract. Ulcerative colitis is localized to the colon and rectum, and affects cells of the mucosa only. Provided herein are methods of treating patients suffering from inflammatory bowel disease comprising administering to a patient a disclosed antisense oligonucleotide.

Inflammatory bowel disease is associated with symptoms including abdominal pain, vomiting, diarrhea, rectal bleeding, severe cramps, muscle spasms, weight loss, malnutrition, fever, anemia, skin lesions, joint pain, eye inflammation, liver disorders, arthritis, pyoderma gangrenosum, primary sclerosing cholangitis, and non-thyroidal illness syndrome, and treating these symptoms using a disclosed antisense compound is also contemplated in an embodiment, for example, treating children suffering from ulcerative colitis who may also suffer from growth defects. In some embodiments, contemplated herein are methods of, e.g., ameliorating or treating such symptoms by administering to a patient an effective amount of a disclosed antisense oligonucleotide.

“Necroptosis,” as used herein, refers to a regulated, caspase-independent cell death, that can be an alternative way to eliminate apoptosis-resistant cancer cells. The core necroptotic pathway consisting of a receptor-interacting protein kinase 1 (RIP1 or RIPK1)—receptor-interacting protein kinase 3 (RIP3 or RIPK3)—mixed lineage kinase domain-like protein (MLKL) complex, also called the ‘necrosome’. The necrosome initiates downstream effector functions such as generation of a reactive oxygen species (ROS) burst, plasma membrane permeabilization, and cytosolic ATP reduction that further drives irreversible necroptosis-executing mechanisms. Provided herein are methods of treating patients by modulating necroptosis comprising administering to a patient a disclosed antisense oligonucleotide.

“A patient in need,” as used herein, refers to a patient suffering from any of the symptoms or manifestations of a bowel disease, a patient who may suffer from any of the symptoms or manifestations of a bowel disease, or any patient who might benefit from a method of the disclosure for treating a bowel disease. A patient in need may include a patient who is diagnosed with a risk of developing a bowel disease, a patient who has suffered from a bowel disease in the past, or a patient who has previously been treated for a bowel disease. Of particular relevance are individuals that suffer from a bowel disease associated with increased levels of FMRP expression or activity.

The terms “treat,” “treatment,” “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment,” as used herein, covers any treatment of a disease in a mammal, particularly a human, and includes: (a) preventing the disease from occurring in a subject which may be predisposed to the disease but has not yet been diagnosed as having it; (b) inhibiting the disease, i.e. preventing the disease from increasing in severity or scope; (c) relieving the disease, i.e. causing partial or complete amelioration of the disease; or (d) preventing relapse of the disease, i.e. preventing the disease from returning to an active state following previous successful treatment of symptoms of the disease or treatment of the disease.

“Effective amount,” as used herein, refers to the amount of an agent that is sufficient to at least partially treat or ameliorate symptoms of a condition when administered to a patient. The effective amount will vary depending on the severity of the condition, the route of administration of the component, and the age, weight, etc. of the patient being treated. Accordingly, an effective amount of a disclosed FMRP antisense oligonucleotide is the amount of the FMRP antisense oligonucleotide necessary to treat a bowel disease in a patient such that administration of the agent to the patient prevents a bowel disease from occurring in a subject, prevents bowel disease progression (e.g., prevents the onset or increased severity of symptoms of a bowel disease such as rectal bleeding, anemia, or gastrointestinal inflammation), or relieves or completely ameliorates all associated symptoms of a bowel disease, i.e. causes regression of the disease.

In some embodiments, disclosed methods include administering to a patient at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of the antisense oligonucleotide. In some embodiments, methods of the disclosure include administering to a patient from 35 mg to 500 mg, from 1 mg to 10 mg, from 10 mg to 20 mg, from 20 mg to 30 mg, from 30 mg to 40 mg, from 40 mg to 50 mg, from 50 mg to 60 mg, form 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of the antisense oligonucleotide.

Efficacy of treatment may be assessed by means of evaluation of gross symptoms associated with a bowel disease, analysis of tissue histology, biochemical assay, imaging methods such as, for example, magnetic resonance imaging, or other known methods. For instance, efficacy of treatment may be evaluated by analyzing gross symptoms of the disease such as changes in abdominal pain, vomiting, diarrhea, rectal bleeding, cramps, muscle spasms, weight loss, malnutrition, fever, anemia or other aspects of gross pathology associated with a bowel disease following administration to the patient of a disclosed FMRP antisense oligonucleotide to a patient suffering from a bowel disease.

Efficacy of treatment may also be evaluated at the tissue or cellular level, for example, by means of obtaining a tissue biopsy (e.g., a tumor or gastrointestinal tissue biopsy) and evaluating gross tissue or cell morphology or staining properties. Biochemical assays that examine protein or RNA expression may also be used to evaluate efficacy of treatment. For instance, one may evaluate FMRP, caspases (e.g., caspase 3 or caspase 8), RIPK1, phospho-RIPK1, RIPK3, phospho-RIPK3, MLKL, phospho-MLKL, CREB, IL-6, IL-8, TNF-alpha, or levels of another protein or gene product indicative of colorectal cancer, necroptosis, inflammatory bowel disease, or inflammatory cytokine production via immunocytochemical, immunohistochemical, Western blotting, or Northern blotting methods, or methods useful for evaluating RNA levels such as quantitative or semi-quantitative polymerase chain reaction. One may also evaluate the presence or level of expression of useful biomarkers found in fecal matter, plasma, or serum to evaluate disease state and efficacy of treatment.

In evaluating efficacy of treatment, suitable controls may be chosen to ensure a valid assessment. For instance, one can compare symptoms evaluated in a patient with a bowel disease following administration to the patient of a disclosed FMRP antisense oligonucleotide to those symptoms in the same patient prior to treatment or at an earlier point in the course of treatment or in another patient not diagnosed with the bowel disease. Alternatively, one may compare the results of biochemical or histological analysis of bowel tissue following administration to the patient of a disclosed FMRP antisense oligonucleotide with those of bowel tissue from the same patient or from an individual not diagnosed with the bowel disease or from the same patient prior to administration to the patient of the FMRP antisense oligonucleotide. Additionally, one may compare blood, serum, cell, or fecal samples following administration to the patient of the FMRP antisense oligonucleotide with comparable samples from an individual not diagnosed with the bowel disease or from the same patient prior to administration to the patient of the FMRP antisense oligonucleotide.

Validation of FMRP inhibition may be determined by direct or indirect assessment of FMRP expression levels or activity. For instance, biochemical assays that measure FMRP protein or RNA expression may be used to evaluate overall FMRP inhibition. For instance, one may measure FMRP protein levels in bowel tissue by Western blot to evaluate overall FMRP levels. One may also measure FMRP mRNA levels by means of Northern blot or quantitative polymerase chain reaction to determine overall FMRP inhibition. One may also evaluate FMRP protein levels or levels of another protein indicative of FMRP activity/expression in dissociated cells, non-dissociated tissue, or fecal matter via immunocytochemical or immunohistochemical methods.

FMRP inhibition may also be evaluated indirectly by measuring parameters such as enhanced expression of RIPK1 and/or activation of the RIPK1-RIPK3-MLKL complex, which are associated with necroptosis. For example, one may measure phospho-RIPK1, phospho-RIPK3, or phospho-MLKL levels in bowel tissue by Western blot.

FMRP down-regulation may also be evaluated indirectly by measuring parameters such as CREB expression. For instance, biochemical assays that measure CREB protein or RNA expression may be used to evaluate overall FMRP inhibition. For instance, one may measure CREB protein levels in bowel tissue by Western blot. One may also measure CREB mRNA levels by means of Northern blot or quantitative polymerase chain reaction to determine overall FMRP inhibition. One may also evaluate CREB protein levels or levels of another protein indicative of CREB activity/expression in dissociated cells, non-dissociated tissue, or fecal matter via immunocytochemical or immunohistochemical methods.

The disclosure provides methods for treating colon cancer. It is contemplated that treatment of colon cancer results in one or more than one of the following: e.g., complete amelioration of disease; reduced number and/or grade of tumors; reduced metastasis; reduced recurrence; and reduced occurrence or severity of symptoms (e.g., diarrhea, constipation, bloody stool, rectal bleeding, abdominal pain, weakness, fatigue, and weight loss).

The disclosure also provides methods for treating IBD (e.g., Crohn's disease and ulcerative colitis). It is contemplated that treatment of IBD results in one or more than one of the following: e.g., complete amelioration of disease; reduced inflammation, including reduced inflammatory cytokine production and intestinal infiltration of immune cells; restoration of intestinal/mucosal architecture; reduced recurrence; and reduced occurrence or severity of symptoms (e.g., diarrhea, constipation, bloody stool, bleeding, abdominal pain, weakness, fatigue, and weight loss). “Inflammatory cytokine production” refers to the expression of cytokines that initiate and/or promote an inflammatory cytokine response. An “inflammatory cytokine response” refers to an immune response that may be characterized by granulocyte recruitment, lymphocyte recruitment, systemic inflammation (especially of the gastrointestinal tract or a portion or portions thereof), fever, tissue destruction, shock, and/or death. An inflammatory cytokine response may be characterized by binding of individual cytokines to their cognate cell surface receptor and subsequent cascades of intracellular signaling that alter cell functions and gene expression. Inflammatory cytokines include, but are not limited to IL-1, IL-6, IL-8, and TNFα. Expression of inflammatory cytokines may occur in, for example, macrophages, monocytes, lamina propria mononuclear cells, or other cells of the gastrointestinal tract or cells of the immune system. Methods of inhibiting inflammatory cytokine production include methods that reduce expression levels of some or all inflammatory cytokines in a patient suffering from an inflammatory bowel disease. Methods of inhibiting inflammatory cytokine production also include methods that reduce expression levels of some or all inflammatory cytokines in cells of a patient suffering from an inflammatory disease.

The disclosure also provides methods of inhibiting FMRP in cells of a patient suffering from a bowel disease. FMRP may be inhibited in any cell in which FMRP expression or activity occurs, including cells of the gastrointestinal tract, immune system, and blood. Cells of the gastrointestinal tract (including cells of the stomach, duodenum, jejunum, ileum, colon, rectum and anal canal), include columnar epithelial cells, mucosal epithelial cells, zymogenic cells, neck mucus cells, parietal cells, gastrin cells, goblet cells, paneth cells, oligomucus cells, and villus absorptive cells. Cells of the immune system include leukocytes, phagocytes (e.g., macrophages, neutrophils, and dendritic cells), monocytes, mast cells, eosinophils, basophils, natural killer cells, innate cells, lymphocytes, B cells, and T cells. Blood cells include red blood cells (erythrocytes) and white blood cells (leukocytes, monocytes, and platelets).

### Tumor Invasion and Tumor Metastasis

Tumor invasion refers to the proliferation of cancerous cells and increase in tumor size leading to extension, breach, penetration, and spread of cancer cells into surrounding tissues. Tumor metastasis occurs when cancer cells break away from a primary tumor site, travel through blood or lymph, and form a new tumor locus in other organs and tissues in the body.

In some embodiments, an effective amount of an FMRP antisense oligonucleotide of the present disclosure can be administered to a patient in need thereof to treat a solid tumor, tumor invasion, or tumor metastasis. In some embodiments, an effective amount of an FMRP antisense oligonucleotide of the present disclosure can be used to prevent or ameliorate tumor invasion or tumor metastasis. In certain embodiments, an effective amount of an FMRP antisense oligonucleotide of the present disclosure can be used to prevent or ameliorate colorectal cancer tumor invasion or colorectal cancer tumor metastasis.

### Pharmaceutical Compositions and Routes of Administration

The present disclosure also provides methods for treating a bowel disease via administration to a patient of a pharmaceutical composition comprising a disclosed FMRP antisense oligonucleotide. In another aspect, the disclosure provides a pharmaceutical composition for use in treating a bowel disease. The pharmaceutical composition may be comprised of a disclosed antisense oligonucleotide that targets FMRP and a pharmaceutically acceptable carrier. As used herein, the term “pharmaceutical composition” means, for example, a mixture containing a specified amount of a therapeutic compound, e.g., an effective amount, of a therapeutic compound in a pharmaceutically acceptable carrier to be administered to a mammal, e.g., a human, in order to treat a bowel disease. In some embodiments, contemplated herein are pharmaceutical compositions comprising a disclosed FMRP antisense oligonucleotide and a pharmaceutically acceptable carrier. In another aspect, the disclosure provides use of a disclosed FMRP antisense oligonucleotide in the manufacture of a medicament for treating an inflammatory disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment, the pharmaceutical composition is administered to a patient orally and includes an enteric coating suitable for regulating the site of absorption of the encapsulated substances within the digestive system or gut. For example, an enteric coating can include an ethylacrylate-methacrylic acid copolymer.

In some embodiments, a disclosed FMRP antisense oligonucleotide and any pharmaceutical composition thereof may be administered to a patient by one or several routes, including enteral or parenteral delivery, or intratumoral injection. As used herein, enteral or enteric administration or delivery refers to administration to a patient of a disclosed FMRP antisense oligonucleotide via the gastrointestinal tract and may include oral, sublingual, gastric and rectal delivery. As used herein, parental administration refers to administration of a disclosed FMRP antisense oligonucleotide to a patient via routes other than the gastrointestinal tract and includes, but is not limited to, intravenous, intratumoral, intranasal, transdermal, subcutaneous, intramuscular, intraperitoneal, intraintestinal (e.g., intrajejunal, intraileal), intracolonic, or intrarectal injections or infusions.

For example, a disclosed FMRP antisense oligonucleotide may be administered to a patient subcutaneously to a subject. In certain examples, a disclosed FMRP antisense oligonucleotide may be administered orally to a subject. In various examples, a disclosed FMRP antisense oligonucleotide may be administered to a patient directly to the gastrointestinal system, or specific regions of the gastrointestinal system (e.g., the jejunum, ileum, colon, or rectum) via parenteral administration.

Pharmaceutical compositions containing a disclosed FMRP antisense oligonucleotide, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, for example, are sterile. Sterilization can be accomplished, for example, by filtration through sterile filtration membranes. Where the composition is lyophilized, filter sterilization can be conducted prior to or following lyophilization and reconstitution.

### Parenteral Administration

The pharmaceutical compositions of the disclosure can be formulated for parenteral administration, e.g., formulated for injection via the intravenous, intratumoral, intramuscular, subcutaneous, intralesional, intraintestinal (e.g., intrajejunal, intraileal), intracolonic, or intrarectal, or intraperitoneal routes. The preparation of an aqueous composition, such as an aqueous pharmaceutical composition containing a disclosed FMRP antisense oligonucleotide, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose, any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed FMRP antisense oligonucleotide may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed FMRP antisense oligonucleotide in the required amount of the appropriate solvent with various amounts of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed FMRP antisense oligonucleotide to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium 10 carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

### Enteral Administration

In some embodiments, contemplated herein are compositions suitable for oral, sublingual, gastric, or rectal delivery of a disclosed FMRP antisense oligonucleotide.

For instance, compositions comprising a disclosed FMRP antisense oligonucleotide may be suitable for oral delivery, e.g., tablets that include an enteric coating, e.g., a gastro-resistant coating, such that the compositions may deliver the FMRP antisense oligonucleotide to, e.g., the gastrointestinal tract of a patient. For example, such administration to a patient may result in a topical effect, substantially topically applying the FMRP antisense oligonucleotide directly to an affected portion of the gastrointestinal tract of a patient. Such administration to a patient, may, in some embodiments, substantially avoid unwanted systemic absorption of the FMRP antisense oligonucleotide.

For example, a tablet for oral administration is provided that comprises granules (e.g., is at least partially formed from granules) that include a disclosed FMRP antisense oligonucleotide, e.g., an antisense oligonucleotide represented by any one of SEQ ID NOs: 1 to 10, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed FMRP antisense oligonucleotide, e.g., an antisense oligonucleotide represented by any one of SEQ ID NOs: 1 to 10, a pharmaceutically acceptable salt, and/or a pharmaceutically acceptable filler. For example, a disclosed FMRP antisense oligonucleotide and a filler may be blended together, optionally, with other excipients, and formed into granules. In some embodiments, the intragranular phase may be formed using wet granulation, e.g., a liquid (e.g., water) is added to the blended FMRP antisense oligonucleotide compound and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Formulations, e.g., that include an intragranular phase and/or an extragranular phase, may include a disintegrant such as, but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed FMRP antisense oligonucleotide and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a formulation may include a lubricant, e.g., an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g. about 0.5% to about 10%, or about 1% to about 20%, by weight of a disclosed FMRP antisense oligonucleotide or a pharmaceutically acceptable salt thereof. Such a tablet may include for example, about 0.5% to about 60% by weight of mannitol, e.g. about 30% to about 50% by weight mannitol, e.g. about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed FMRP antisense oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In various embodiments, a pharmaceutical tablet formulation for oral administration of a disclosed FMRP antisense oligonucleotide comprises an intra-granular phase, wherein the intra-granular phase includes a disclosed FMRP antisense or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed FMRP antisense oligonucleotide, e.g., a disclosed FMRP antisense oligonucleotide or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In some examples, a pharmaceutically acceptable tablet for oral use may comprise an intra-granular phase, comprising about 5 to about 10% by weight of a disclosed FMRP antisense oligonucleotide, e.g., a disclosed FMRP antisense oligonucleotide or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments, the pharmaceutical composition may contain an enteric coating comprising about 13% or about 15%, 16%, 17% or 18% by weight, e.g., Acryl-EZE® (see, e.g., PCT Publication No. WO2010/054826, which is hereby incorporated by reference in its entirety).

The rate at which point the coating dissolves and the active ingredient is released is its dissolution rate. In some embodiments, a tablet may have a dissolution profile, e.g., when tested in a USP/EP Type 2 apparatus (paddle) at 100 rpm and 37° C. in a phosphate buffer with a pH of 7.2, of about 50% to about 100% of the FMRP antisense oligonucleotide releasing after about 120 minutes to about 240 minutes, for example after 180 minutes. In certain embodiments, a tablet may have a dissolution profile, e.g., when tested in a USP/EP Type 2 apparatus (paddle) at 100 rpm and 37° C. in diluted HCl with a pH of 1.0, where substantially none of the FMRP antisense oligonucleotide is released after 120 minutes. A tablet, in some embodiments, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the FMRP antisense oligonucleotide releasing after 30 minutes.

Formulations, e.g., tablets, in some embodiments, when orally administered to the patient may result in minimal plasma concentration of the FMRP antisense oligonucleotide in the patient. In another embodiment, disclosed formulations, when orally administered to a patient, topically deliver to the colon or rectum of a patient, e.g., to an affected or diseased site of a patient.

Administration to a patient of a disclosed FMRP antisense oligonucleotide may occur via the rectal mode. Pharmaceutical compositions for rectal administration include foams, solutions (enemas), and suppositories (Block, Chapter 87 In: Remington's Pharmaceutical Sciences, 18th Ed., Gennaro, ed., Mack Publishing Co., Easton, Pa., 1990).

In some embodiments, methods provided herein may further include administering to a patient at least one other agent that is directed to treatment of diseases and disorders disclosed herein. In some embodiments, contemplated other agents may be co-administered (e.g., sequentially or simultaneously) to a patient.

For example, such agents include, but are not limited to, chemotherapeutic agents, such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, vincristine, vinblastine, etoposide, trimetrexate, teniposide, cisplatin and diethylstilbestrol (DES).

Agents also include immunosuppressive agents including glucocorticoids, cytostatics, antibodies, agents acting on immunophilins, interferons, opioids, TNF binding proteins, mycophenolate, and small biological agents. For example, contemplated immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, certolizumab pegol, natalizumab, and etanercept. Other agents include antibiotics, anti-diarrheals, laxatives, pain relievers, iron supplements, and calcium or vitamin D or B-12 supplements.

### Dosage and Frequency of Administration

Exemplary formulations include dosage forms that include or consist essentially of about 35 mg to about 500 mg of a disclosed FMRP antisense oligonucleotide. For example, formulations that include about 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, or 250 mg of a disclosed FMRP antisense oligonucleotide are contemplated herein. In one embodiment, a formulation may include about 40 mg, 80 mg, or 160 mg of a disclosed FMRP antisense oligonucleotide. In some embodiments, a formulation may include at least 100 μg of a disclosed FMRP antisense oligonucleotide. For example, formulations may include about 0.1 mg, 0.2 mg, 0.3 mg, 0.4 mg, 0.5 mg, 1 mg, 5 mg, 10 mg, 15 mg, 20 mg, or 25 mg of a disclosed FMRP antisense oligonucleotide. The amount administered to a patient will depend on variables such as the type and extent of disease or indication to be treated, the overall health and size of the patient, the in vivo potency of the FMRP antisense oligonucleotide, the pharmaceutical formulation, and the route of administration. The initial dosage can be increased beyond the upper level in order to rapidly achieve the desired blood-level or tissue level. The initial dosage can be smaller than the optimum, and the dosage may be progressively increased during the course of treatment. Human dosage can be optimized, e.g., in a conventional Phase I dose escalation study designed to run from 40 mg to 160 mg. Dosing frequency can vary, depending on factors such as route of administration, dosage amount and the disease being treated. Exemplary dosing frequencies are once per day, once per week and once every two weeks. In some embodiments, dosing is once per day for 7 days.

### Diagnostic Methods

The disclosure also provides a method of diagnosing a patient with a bowel disease that relies upon detecting levels of FMRP expression signal in one or more biological samples of a patient. As used herein, the term “FMRP expression signal” can refer to any indication of FMR1 gene expression, FMR1 gene products, or FMRP activity. FMR1 gene products include RNA (e.g., mRNA), peptides, and proteins (e.g., FMRP, variants, analogs, and/or portions thereof). Indices of FMR1 gene expression that can be assessed include, but are not limited to, FMR1 gene or chromatin state FMR1 gene interaction with cellular components that regulate gene expression, FMR1 gene product expression levels (e.g., FMRP RNA expression levels, FMRP protein expression levels), or interaction of FMRP RNA or protein with transcriptional, translational, or post-translational processing machinery. Indices of FMRP activity include, but are not limited to, assessment of RIPK/MLKL pathway activation (e.g., assessment of RIPK1, RIPK3 and/or MLKL phosphorylation) and CREB expression (e.g., mRNA and protein expression).

Detection of FMRP expression signal may be accomplished through in vivo, in vitro, or ex vivo methods. In a preferred embodiment, methods of the disclosure may be carried out in vitro. Methods of detecting may involve detection in blood, serum, fecal matter, tissue, or cells of a patient. Detection may be achieved by measuring FMRP expression signal in whole tissue, tissue explants, cell cultures, dissociated cells, cell extract, or body fluids, including blood or serum. Contemplated methods of detection include assays that measure levels of FMR1 gene product expression such as Western blotting, flow cytometry, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### EXAMPLES

The disclosure is further illustrated by the following examples. The examples are provided for illustrative purposes only, and are not to be construed as limiting the scope or content of the disclosure in any way.

### Example 1: FMRP is Unregulated in Human Colorectal Cancer

To investigate the role of FMRP in CRC tumorigenesis, the levels of FMRP mRNA and protein levels in human CRC samples were analyzed. This example demonstrates the presence of increased FMRP expression in human CRC samples.

### Patients and Samples

Match-paired samples of human CRC tumors and adjacent macroscopically unaffected areas were taken from 48 patients undergoing colonic resection for sporadic CRC at the Tor Vergata University Hospital (Rome, Italy). None of the patients received radiotherapy or chemotherapy prior to surgery.

### Immunohistochemistry

Immunohistochemistry was performed on formalin-fixed, paraffin-embedded sections of normal tissues and paired tumor and peritumor samples of CRC patients. Colonic sections were deparaffinised and dehydrated using xylene and ethanol and antigen retrieval was performed in Tris-EDTA citrate buffer (pH 7.8) for 30 min. in a thermostatic bath at 98° C. (Dako Agilent Technologies, Glostrup, Denmark). Immunohistochemical staining was performed using a monoclonal antibody directed against human FMRP (final dilution 1:500, LifeSpan BioSciences, Inc.) incubated at room temperature for 1 hour followed by a biotin-free HRP-polymer detection technology with 3,3′diaminobenzidine (DAB) as a chromogen (MACH 4™ Universal HRP-Polymer Kit, Biocare Medical, Pacheco, Calif.). Sections were counter-stained with haematoxylin, dehydrated and mounted. Isotype control IgG-stained sections were prepared under identical immunohistochemical conditions as described above, replacing the primary antibody with a purified mouse normal IgG control antibody (R&D Systems, Minneapolis, Minn.).

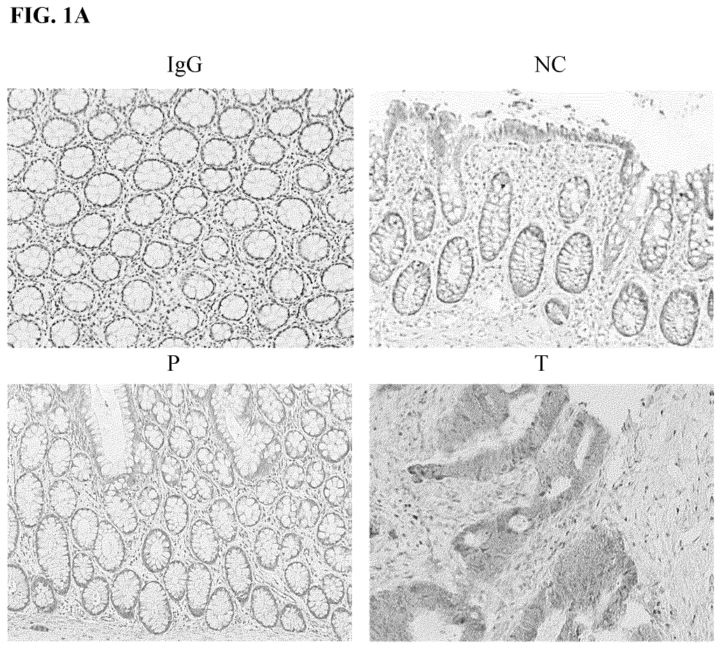

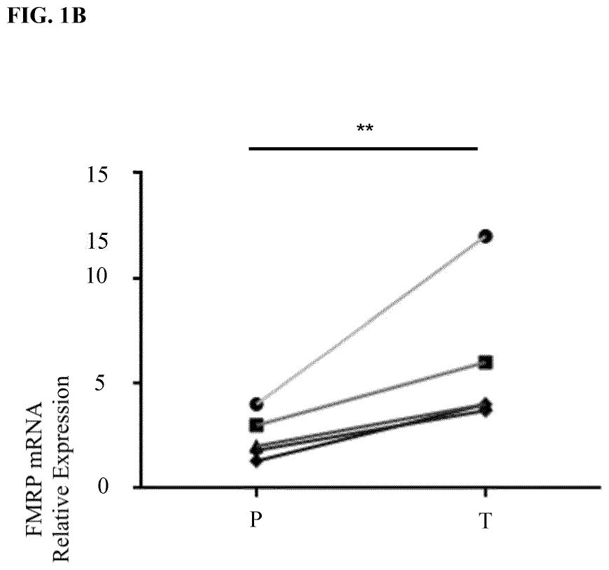

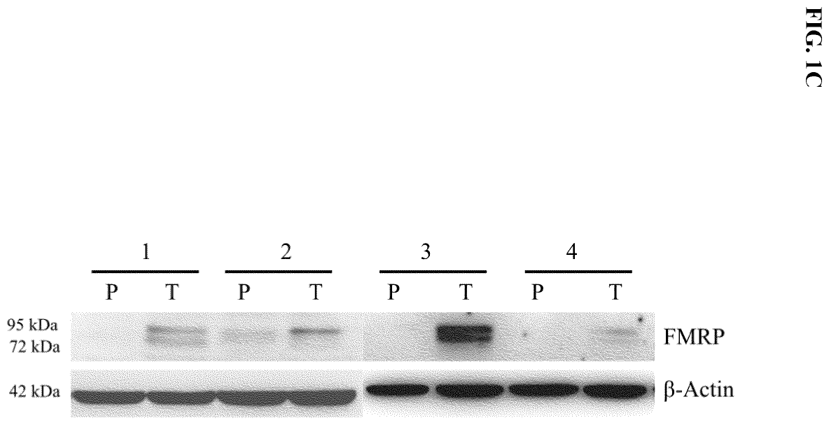

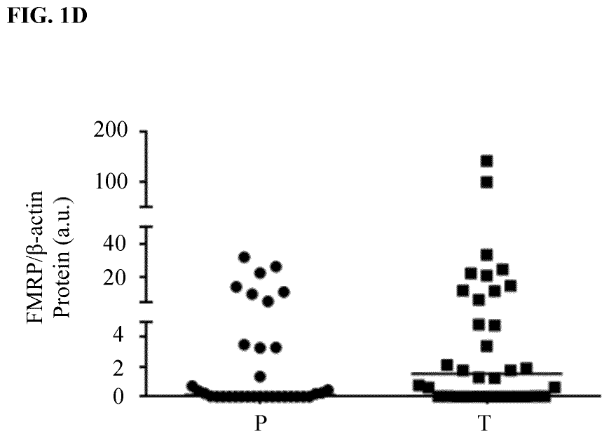

FMRP expression was analysed in sections (n=40) taken from matched pairs of samples of human CRC tumors and adjacent areas, and in samples taken from colons of normal controls (NC). As showin in FIG. 1A, FMRP protein expression was significantly increased in CRC tumor samples (T) compared to peritumor areas (P) and NC. FMRP was strongly expressed in approximately 61% of CRC samples (24/40), as compared to adjacent non-tumor areas and NC, which express relatively lower levels of FMRP.

FMRP mRNA Expression

RNA was extracted from match-paired samples of human CRC and adjacent macroscopically unaffected areas using PureLink® mRNA mini kit (Thermo Fisher Scientific, Waltham, Mass., U.S.A.), according to the manufacturer's instructions. A constant amount of RNA (1 μg per sample) was reverse transcribed into complementary DNA (cDNA) and this was amplified using the following conditions: denaturation for 1 min at 95° C.; annealing for 30 s at 59° C. for human FMRP, 60° C. for human/mouse β-actin; 30 s of extension at 72° C. β-actin was used as a house-keeping gene. Gene expression was calculated using the AACt algorithm. Primer sequences were as follows: human FMRP (forward 5′-GTTGAGCGGCCGAGTTTGTCAG-3′ (SEQ ID NO: 11); reverse 5′-CCCACTGGGAGAGGATTATTTGGG-3′ (SEQ ID NO: 12)), human and mouse β-actin (forward 5′-AAGATGACCCAGATCATGTTTGAGACC-3′ (SEQ ID NO: 13); reverse 5′-AGCCAGTCCAGACGCAGGAT-3′ (SEQ ID NO: 14)).

As seen in FIG. 1B, RT-PCR confirmed the immunohistochemical analysis and showed that FMRP mRNA was significantly up-regulated in human CRC tumor samples (T) compared to peritumor samples (P) which were taken from macroscopically unaffected areas of the same patients.

### FMRP Protein Expression

Total proteins were extracted from human colonic tissues, and lysed on ice in buffer containing 10 mM HEPES [pH 7.9], 10 mM KCl, 0.1 mM EthyleneDiamineTetraacetic Acid (EDTA), 0.2 mM Ethylene Glycol-bis (β-aminoethyl ether)-N,N,N′,N′-Tetraacetic Acid (EGTA) and 0.5% Nonidet P40 supplemented with 1 mM dithiothreitol (DTT), 10 mg/ml aprotinin, 10 mg/ml leupeptin, 1 mM phenylmethylsulfonyl fluoride (PMSF), 1 mM Na₃VO₄, and 1 mM NaF. Lysates were clarified by centrifugation and separated by sodium dodecyl sulphate (SDS)-polyacrylamide gel electrophoresis. Blots were incubated with antibodies against: FMRP (Cell Signaling, Danvers, Mass.), followed by a secondary antibody conjugated to horseradish peroxidase (Dako, Milan, Italy). After analysis, each blot was stripped and incubated with a mouse-anti-human monoclonal β-actin antibody (Sigma-Aldrich) to ascertain equivalent loading of the lanes.

As seen in FIGS. 1C and 1D, Western blot analysis confirmed the immunohistochemical and RT-PCR analysis and showed that FMRP protein was significantly up-regulated in human CRC tumor samples (T) compared to peritumor samples (P) which were taken from macroscopically unaffected areas of the same patients.

### Example 2: FMR1 Knockout Mice are Resistant to Azoxymethane-Induced Tumorigenesis

To dissect the role of FMRP in CRC, a mouse model of sporadic CRC induced by Azoxymethane (AOM) was used. This example describes the tumor-resistant phenotype of FMR1 knockout mice in the AOM-induced CRC model.

### AOM-Induction of CRC

Wild type (WT) and FMR1 knock-out (KO) mice were injected with the alkylating agent, AOM (10 mg/kg; Sigma Aldrich, Milan, Italy), intraperitoneally once a week for 5 weeks in order to induce tumor formation. Mice were monitored for tumor formation and were endoscopically screened using a high-resolution endoscope system 7 days before sacrifice. At week 22, mice were sacrificed by cervical dislocation and colonic tissues were collected for analysis.

Colonoscopies were performed in a blinded manner to monitor tumor formation using a high-resolution mouse endoscope system. Tumors were detected during endoscopic examination, performed at week 21. After termination of treatment, tumors were counted to determine the total number of colonic lesions. All tumors were evaluated based on their size and scored using the protocol described in Becker C. et al., Gut. 2005; 54(7):950-4., herein incorporated by reference in its entirety. In brief, tumors were graded as follows: grade 1 (very small but detectable tumor), grade 2 (tumor covering up to one eighth of the colonic circumference), grade 3 (tumor covering up to a quarter of the colonic circumference), grade 4 (tumor covering up to half of the colonic circumference), and grade 5 (tumor covering more than half of the colonic circumference).

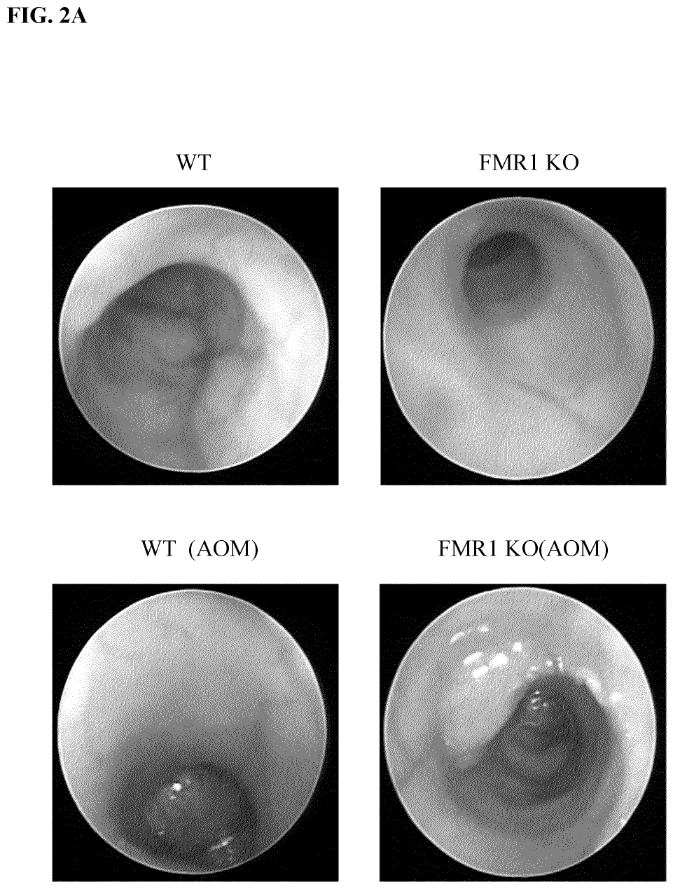

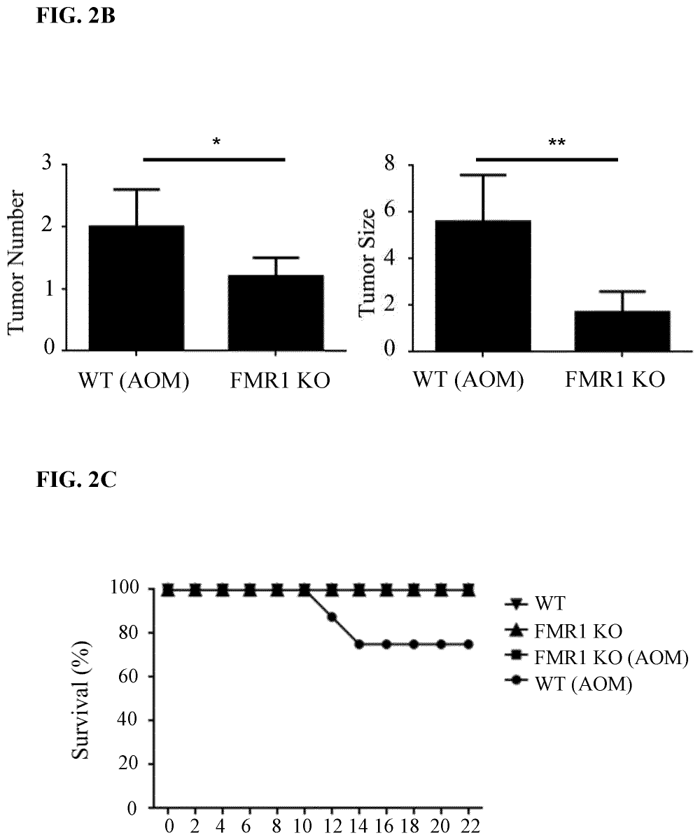

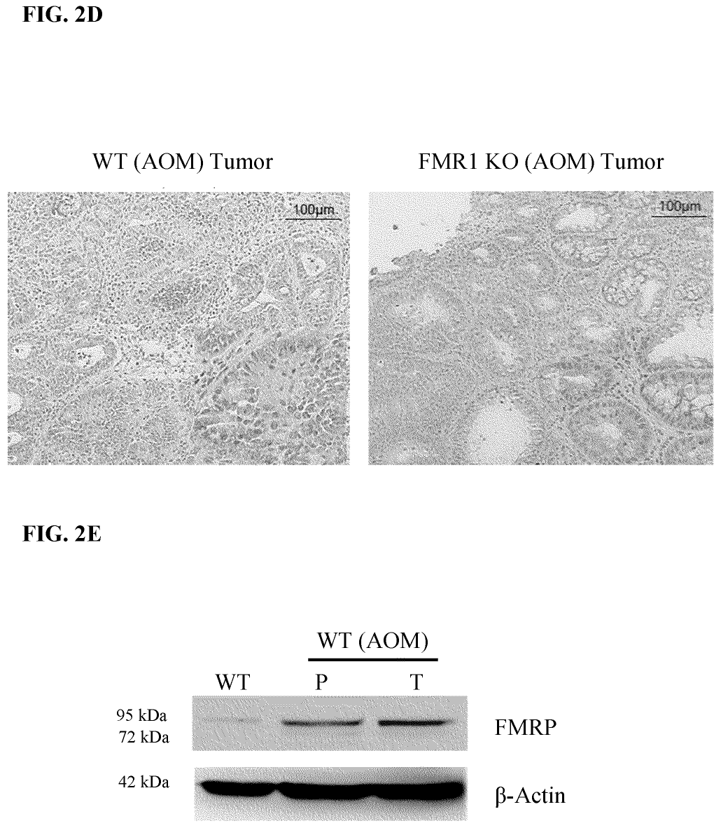

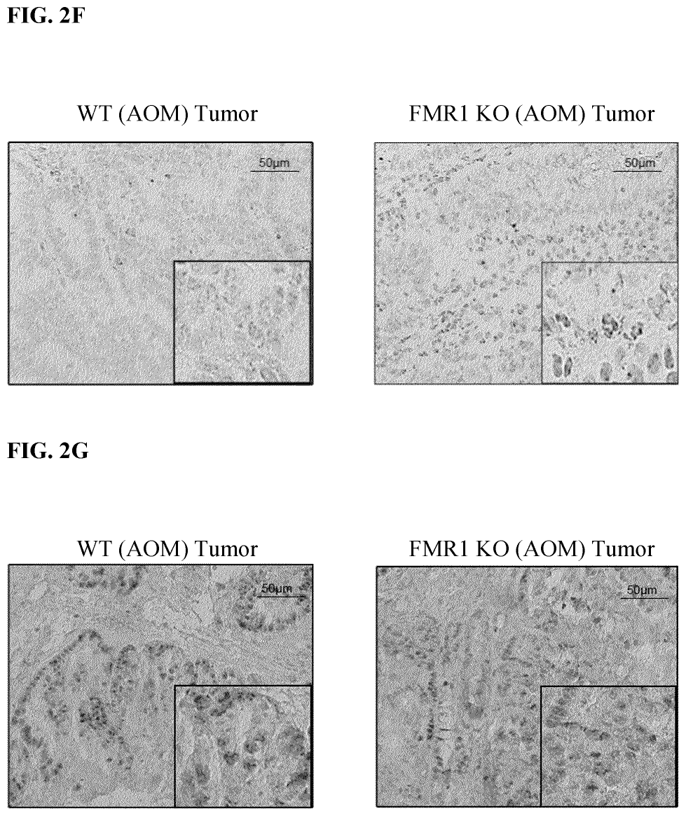

As seen in FIGS. 2A and 2B, WT mice treated with AOM developed multiple large tumors, while the number and size of tumors in AOM-treated FMR1 KO mice were significantly reduced. These results were confirmed by direct assessment of tumors in mice sacrificed on week 22 (data not shown). As shown in FIG. 2C, AOM-treated WT mice exhibited a 20% decrease in viability as compared to AOM-treated FMR1 KO animals.

### Immunohistochemistry

Histological analysis was performed on mouse colonic cryosections taken from WT and FMR1 KO mice in tumor and peritumor areas after hematoxylin and eosin (H&E) staining. Colonic sections were deparaffinised and dehydrated using xylene and ethanol and antigen retrieval was performed in Tris-EDTA citrate buffer (pH 7.8) for 30 min in a thermostatic bath at 98° C. (Dako Agilent Technologies, Glostrup, Denmark). Immunohistochemical staining was performed using a monoclonal antibody directed against human FMRP (final dilution 1:500, LifeSpan BioSciences, Inc.) incubated at room temperature for 1 hour followed by a biotin-free HRP-polymer detection technology with 3,3′diaminobenzidine (DAB) as a chromogen (MACH 4™ Universal HRP-Polymer Kit, Biocare Medical, Pacheco, Calif.). Sections were counter-stained with haematoxylin, dehydrated and mounted. Isotype control IgG-stained sections were prepared under identical immunohistochemical conditions as described above, replacing the primary antibody with a purified mouse normal IgG control antibody (R&D Systems, Minneapolis, Minn.).

In the absence of AOM treatment, the intestines of FMRP-KO mice were normal and there were no macroscopic abnormalities as compared to WT mice (data not shown). However, as shown in FIG. 2D, tumors excised at week 22 post-AOM treatment showed that WT mice developed tumors with pervasive dysplasia, whereas FMR1 KO animals had preserved normal mucosal tissue architecture adjacent to dysplastic areas.

### FMRP Protein Expression

Total proteins were extracted from mouse colonic tissues, and lysed on ice in buffer containing 10 mM HEPES [pH 7.9], 10 mM KCl, 0.1 mM EthyleneDiamineTetraacetic Acid (EDTA), 0.2 mM Ethylene Glycol-bis (β-aminoethyl ether)-N,N,N′,N′-Tetraacetic Acid (EGTA) and 0.5% Nonidet P40 supplemented with 1 mM dithiothreitol (DTT), 10 mg/ml aprotinin, 10 mg/ml leupeptin, 1 mM phenylmethylsulfonyl fluoride (PMSF), 1 mM Na₃VO₄, and 1 mM NaF. Lysates were clarified by centrifugation and separated on sodium dodecyl sulphate (SDS)-polyacrylamide gel electrophoresis. Blots were incubated with antibodies against: FMRP (Cell Signaling, Danvers, Mass.), followed by a secondary antibody conjugated to horseradish peroxidase (Dako, Milan, Italy). After analysis, each blot was stripped and incubated with a mouse-anti-human monoclonal β-actin antibody (Sigma-Aldrich) to ascertain equivalent loading of the lanes.

As shown in FIG. 2E, consistent with human CRC samples, FMRP protein expression was increased in mucosal tumors (T) of AOM-treated WT mice, as compared to adjacent peritumor areas (P) of the same animals.

### TUNEL and Ki67 Staining

To determine whether the decreased tumorigenesis observed in FMR1 KO animals was due to increased cell death or decreased tumor cell proliferation, TUNEL staining and immunohistochemical staining for Ki67 was performed. In colonic cryosections taken from WT and FMR1 KO mice, apoptotic cells were detected using a TUNEL in situ cell death detection kit (Roche Applied Science) according to the manufacturer's instructions. 3-Amino-9-ethylcarbazole was used as a chromogen, and sections were counterstained with hematoxylin. Apoptotic cell nuclei appeared as red-stained structures against a blue-violet background.

Immunohistochemistry sections were also incubated with a mouse monoclonal antibody directed against mouse Ki67 (clone MIB-5, final dilution 1:100, DaKO, Agilent, Santa Clara, Calif., USA) at room temperature for 30 minutes, followed by biotin-free HRP polymer detection (Ultravision Detection System, Thermo Scientific, Waltham, Mass., USA) with 3,3′diaminobenzidine as a chromogen (DaKO, Agilent). The sections were counterstained with haematoxylin, dehydrated, and mounted.

As shown in FIG. 2F, TUNEL staining revealed that AOM-treated FMR1 KO mice exhibited increased levels of DNA fragmentation as compared to AOM-treated WT animals. However, as seen in FIG. 2G, there were no differences observed in the number of Ki67-positive cells. Collectively, these results suggest that FMRP is associated with increased tumor resistance to cell death.

### Example 3: FMRP Antisense Oligonucleotides Induce Cell Death in CRC Cell Lines

To determine how FMRP affects CRC survival, cell death in human CRC epithelial cell lines were treated with a specific FMRP antisense oligonucleotide. This example demonstrates that FMRP antisense oligonucleotides can induce cell death in CRC cell lines via an apoptosis-independent mechanism.

### FMRP Expression in Colorectal Cancer Cell Cultures

Human CRC cell lines, DLD-1 and HCT-116, were obtained from the American Type Culture Collection (ATCC, Manassas, Va.) and cultured in RPMI 1640 (DLD-1) and McCoy's 5A (HCT-116) medium, respectively. All media were supplemented with 10% fetal bovine serum, 1% penicillin/streptomycin (both from Lonza, Verviers, Belgium). The normal human colon epithelial cell line (HCEC-1ct) was obtained from EVERCYTE GmbH (Vienna, Austria) and cultured in ColoUp® medium (EVERCYTE GmbH). Cells were maintained in a 37° C., 5% CO2, fully humidified incubator. Cell line lysates were prepared and analyzed by SDS-polyacrylamide gel electrophoresis/immunoblot using methods as previously described in Example 2. For immunofluorescent staining, cell lines were fixed with 3.7% formaldehyde for 10 minutes at 4° C., permeabilized with 0.1% Triton for 10 minutes at room temperature and blocked (1% bovine serum albumin, Tween 0.1%, glycine 2%) for 1 hour at room temperature. Fixed and blocked cells were incubated with anti-FMRP monoclonal antibody (1:500, Cell Signaling, Danvers, Mass.) overnight at 4° C. After washing with PBS, secondary antibody goat anti-rabbit Alexa 488 (1:2000, A11008; Invitrogen) was applied for 1 hour at room temperature. Slides were washed with PBS, mounted using Prolong Gold® anti-fade reagent with 4′,6-diamidino-2-phenylindole (P36931; Invitrogen) and analyzed by a Leica DMI4000 B microscope with Leica application suite software (V4.6.2).

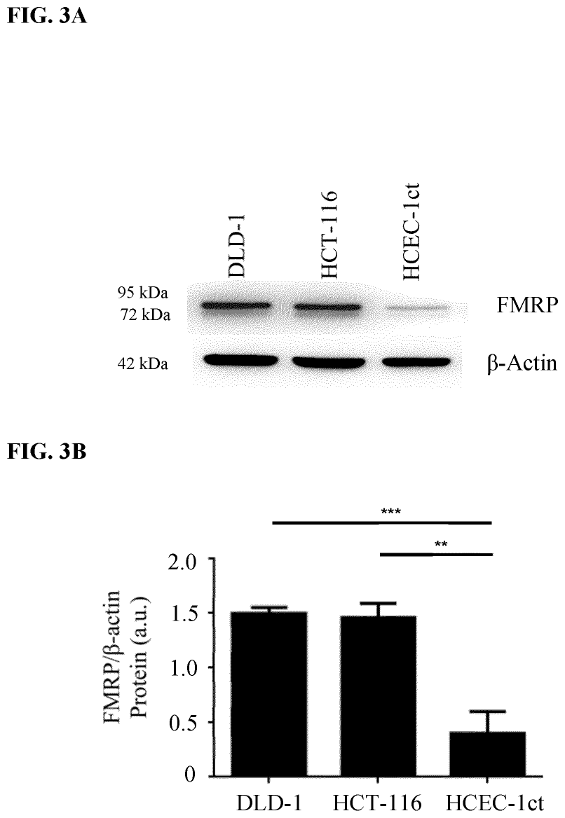

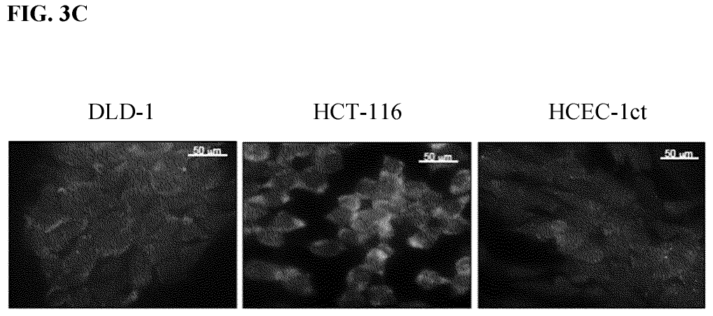

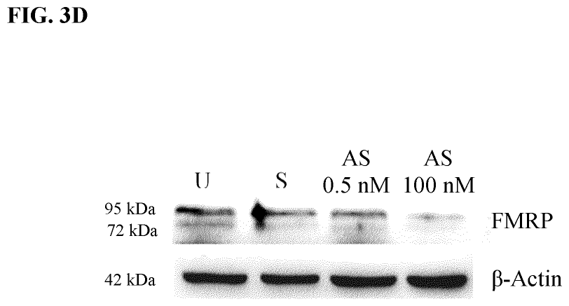

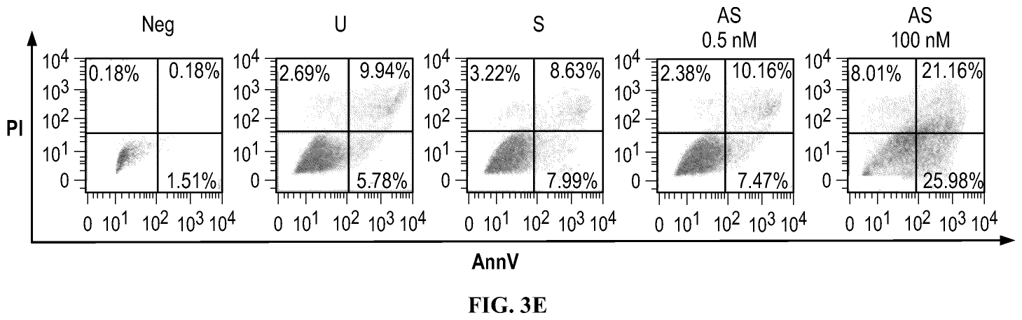

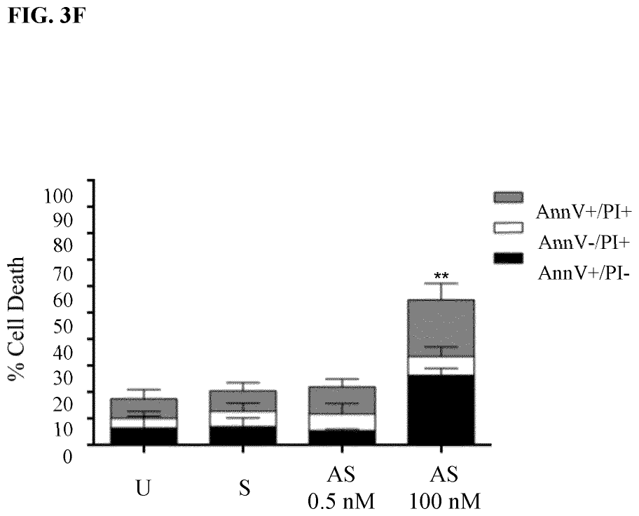

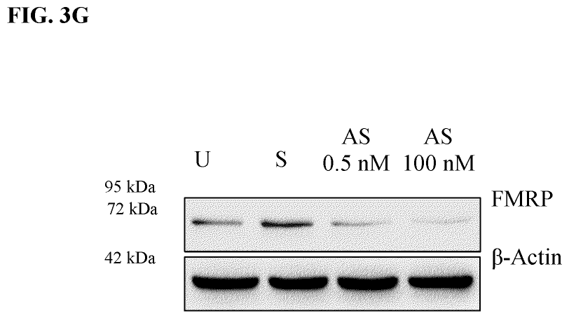

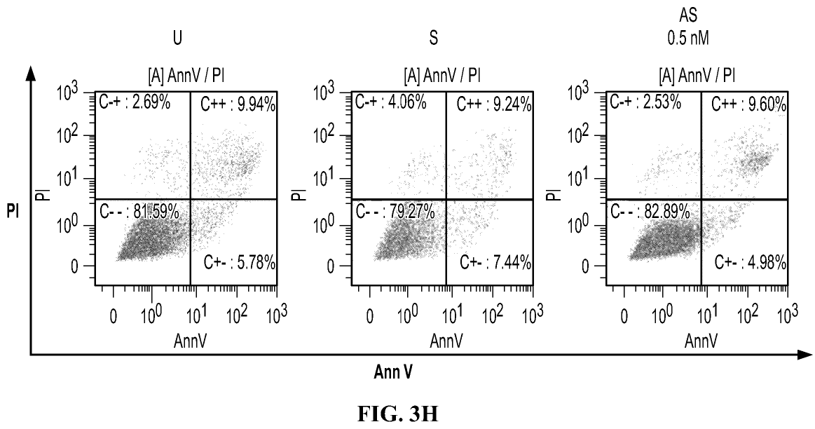

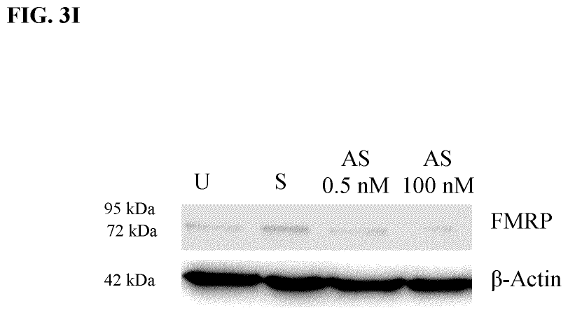

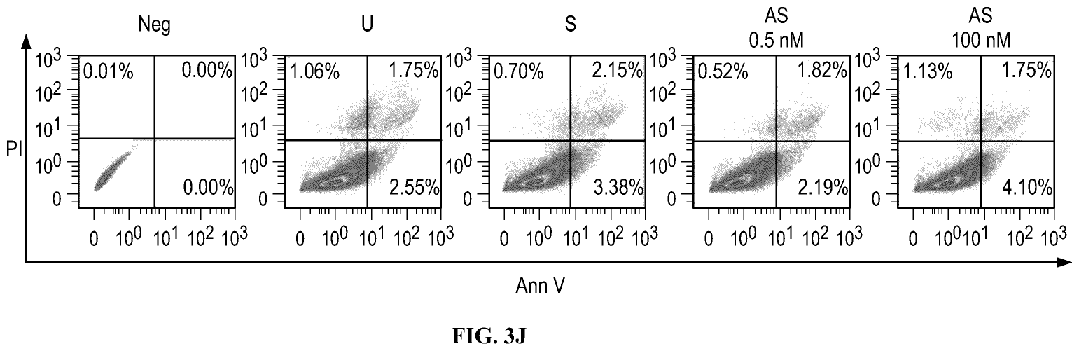

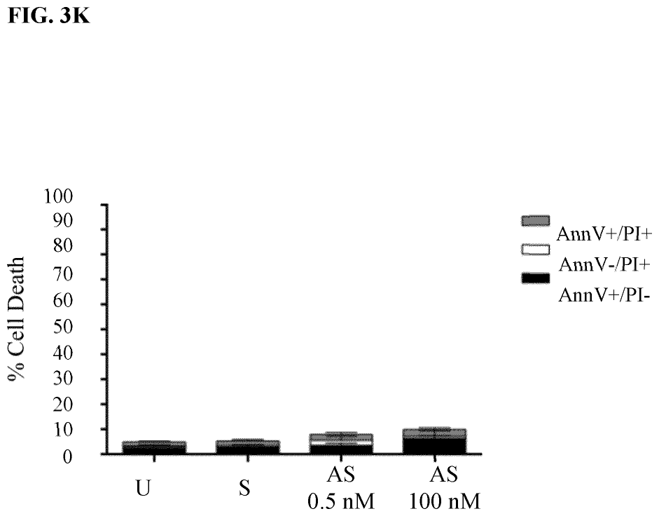

As shown in FIGS. 3A-3C, FMRP was more highly expressed in CRC human cell lines (DLD-1 and HCT-116) as compared to HCEC-1ct epithelial cells (i.e. normal colon cells).

### FMRP Knockdown in Colorectal Cancer Cell Cultures

Phosphorothioate single-stranded antisense oligonucleotides complementary to human FMRP [5′-TCCACCACCAGCTCCTCCAT-3′ (SEQ ID NO: 6)] and single-stranded sense oligonucleotides [5′-ATGGAGGAGCTGGTGGTGGA-3′ (SEQ ID NO: 15)] were synthesized. CRC cell lines and HCEC-1ct cells were transfected with either FMRP antisense (AS)(final concentration 0.5-100 nM) or FMRP sense oligonucleotide (S) (final concentration100 nM) for 24 and 48 hours, using Opti-MEM medium and lipofectamine 3000 reagent (Thermo Fisher Scientific, Waltham, Mass., USA) according to the manufacturer's instructions.

As seen in FIGS. 3D, 3G, and 3I, treatment of DLD-1 cells (FIG. 3C), HCT-116 cells (FIG. 3G), and HCEC-1ct cells (FIG. 3I) with FMRP antisense oligonucleotide (AS) significantly inhibited FMRP expression and no significant inhibition was observed in cells treated with sense oligonucleotide.

### Cell Death in FMRP Antisense Oligonucleotide Treated Cells.

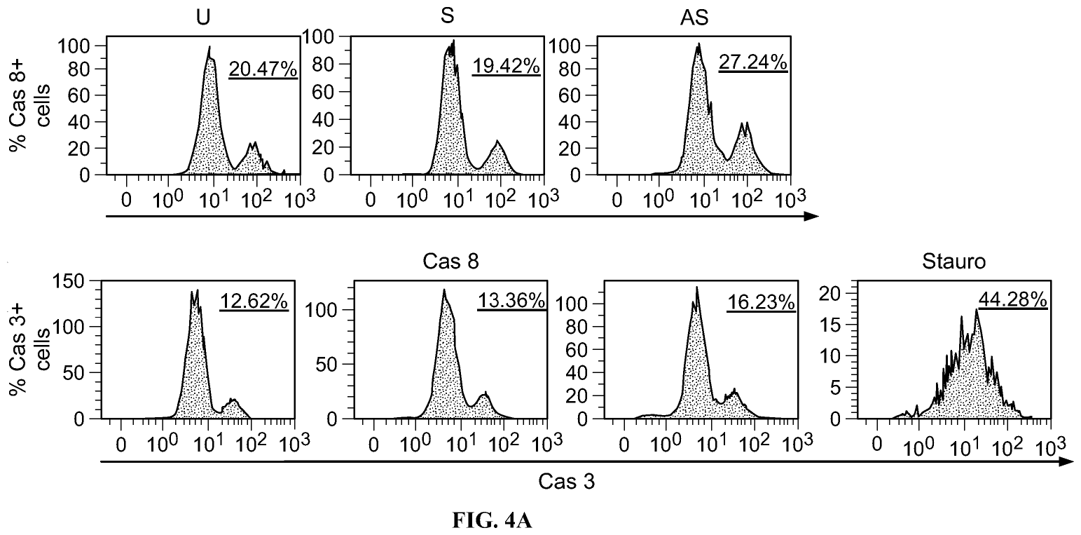

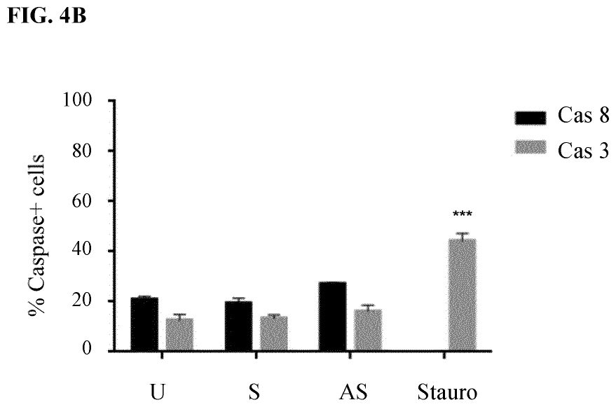

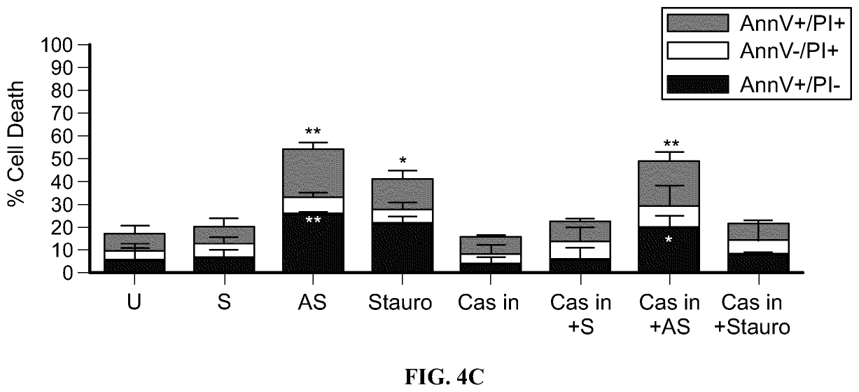

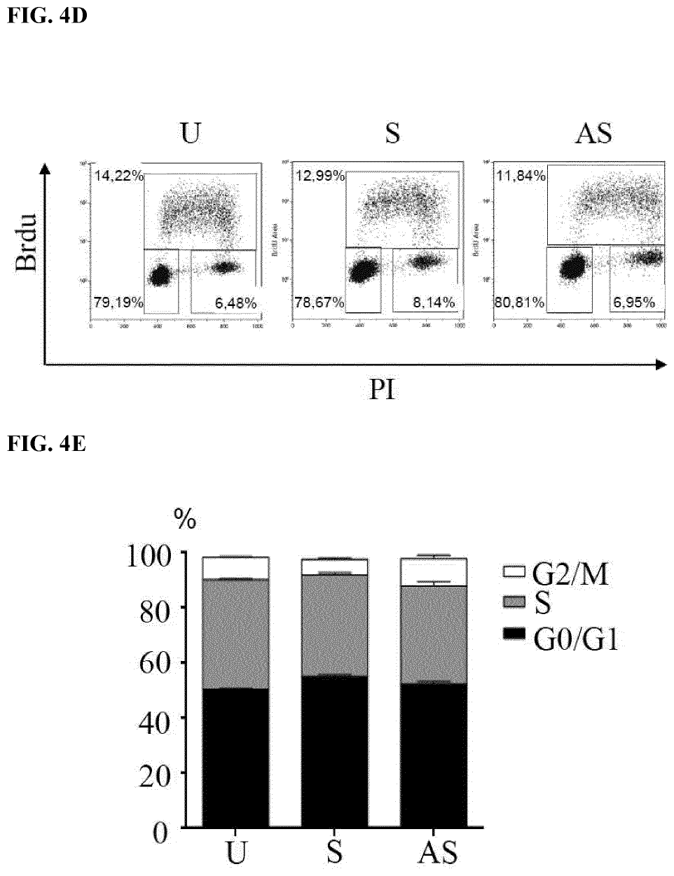

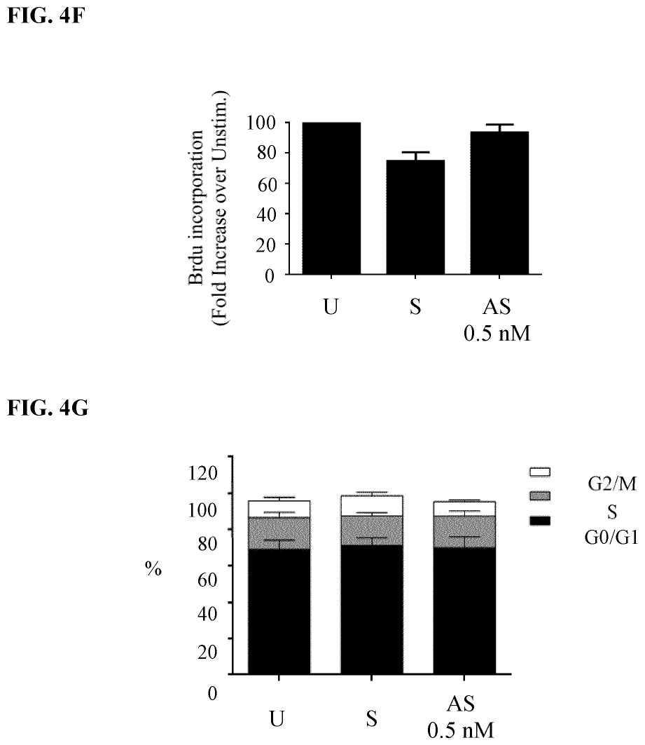

Cells were transfected with either FMRP antisense oligonucleotide (AS)(final concentration 0.5 nM and 100 nM) or FMRP sense oligonucleotide (S)(final concentration 100 nM). After 24 hours (FIGS. 3E, 3F, 3J and 3K) or 48 hours (FIG. 3H), cells were collected, washed two times in Annexin V (AV) buffer, stained with FITC-AnnexinV (final dilution 1:100; Immunotools, Friesoythe, Germany) according to the manufacturer's instructions, and incubated with 5 mg/ml of propidium iodide (PI) for 30 min at 4° C. Cells were also transfected with either FMRP sense oligonucleotide or FMRP AS oligonucleotide (final concentration 100 nM) for 36 hours and analyzed for activated caspase 3 and caspase 8. As positive and negative controls, cells were treated, respectively, with staurosporin (final concentration 1 μM, Sigma-Aldrich Milan, Italy) or Q-VD-OPh (a pan-caspase inhibitor; final concentration 1 μM) (R&D Systems, Inc, Minneapolis, Minn.). Additionally, 36 hours (FIGS. 4F and 4G) or 48 hours post-transfection (FIGS. 4D and 4E), cells were pulsed with 10 M bromodeoxyuridine for 60 minutes, fixed in 70% cold ethanol, and stored at −20° C. for at least 3 hours. Cells were then denatured in 2 M HCl, and stained with anti-bromodeoxyuridine monoclonal antibody (Immunotech, Marseille, France) followed by fluorescein isothiocyanate-conjugated secondary anti-mouse immunoglobulin G (Molecular Probes, Milan, Italy) and 100 g/mL PI. Fluorescence was measured using Gallios Flow Cytometer (Beckman Coulter, Life Sciences, Pasadena, Calif., USA) and analyzed using Kaluza software (Beckman Coulter). Viable cells were considered as AV⁻/PI⁻ cells.

As shown in FIGS. 3E and 3F, treatment of DLD-1 cells with FMRP antisense oligonucleotide resulted in an increased number of AnnexinV⁺ or AnnexinV⁺ Pt cells indicating that these CRC cells were undergoing spontaneous apoptosis or necroptosis. However, as shown in FIGS. 3H, 3J and 3K, neither HCT-116 cells nor HCRC-1ct cells treated with FMRP antisense oligonucleotide exhibited an increased number of AnnexinV⁺ or AnnexinV⁺ P⁺ cells. Similar results were observed using a specific FMRP siRNA transfected into cell lines (data not shown).

To dissect the mechanisms of FMRP antisense oligonucleotide-induced cell death, the effect of FMRP knockdown on activation of pro-apoptosis caspase 8 and caspase 3 was analyzed. As shown in FIGS. 4A and 4B, treatment of CRC cells with FMRP antisense oligonucleotide did not alter the percentage of activated caspase 3-positive or activated caspase 8-positive cells. As expected, staurosporin (Stauro) significantly increased the percentage of activated caspase 3 positive cells.

As seen in FIG. 4C, pre-treatment of cells with a pan-caspase inhibitor (Q-VD-OPh; Cas in) did not alter FMRP antisense oligonucleotide-induced cell death. Moreover, to verify whether FMRP antisense oligonucleotide-induced cell death was secondary to cell growth arrest, cell cycle progression in DLD-1 cell lines was analyzed. As shown in FIGS. 4D-4G, FMRP antisense oligonucleotide did not affect the relative percentage of cells in G2/M, S, or G0/G1 cell cycle phases before the induction of cell death. Together, these findings suggest that FMRP influences CRC cell death via an apoptosis-independent pathway that does not affect the cell cycle.

### Example 4: FMRP Antisense Oligonucleotides Induce Necroptosis

Cancer cells have developed a variety of mechanisms to escape programmed cell death. Necroptosis is a regulated, caspase-independent, cell death pathway that is an alternative mechanism for eliminating apoptosis-resistant cells. This example demonstrates that FMRP anti sense oligonucleotides induce cell death in CRC cells via activation of the necroptotic pathway.

FMRP Associates with mRNAs Associated with the Necroptosis Pathway

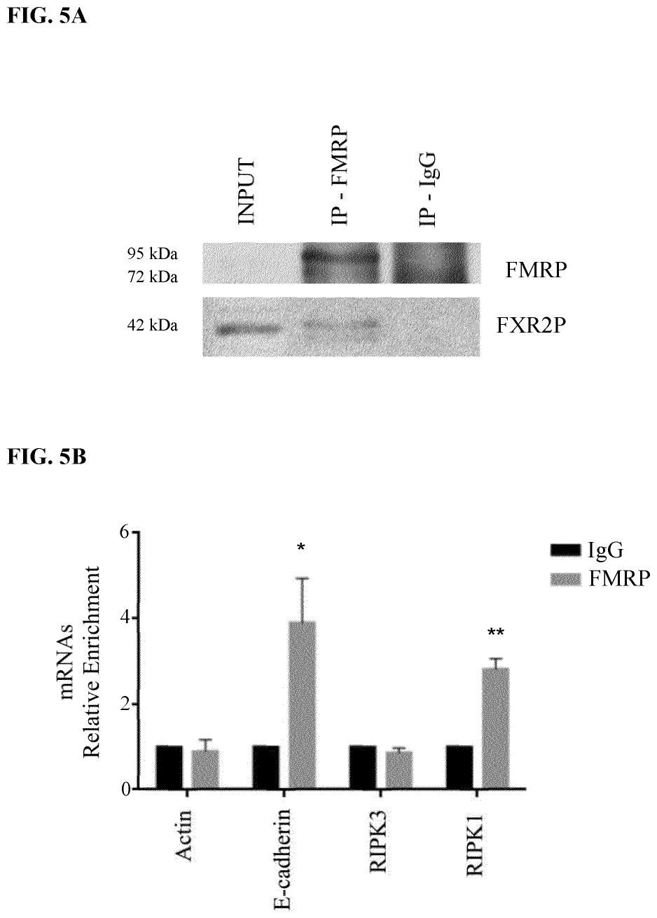

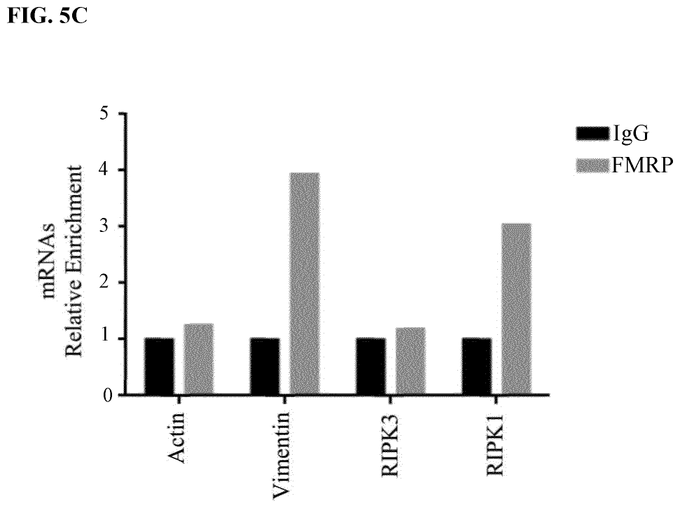

FMRP from human CRC samples and CRC cell lines, was immunoprecipitated together with its associated RNA using a FMRP-specific antibody (FIG. 5A) for identification of bound transcripts by real-time PCR. β-actin was used as a negative control, and E-cadherin and vimentin were used as positive controls. As shown in FIGS. 5B and 5C, RIPK3 mRNA does not co-immunoprecipitate with FMRP in human CRC samples or CRC cell lines. In contrast, significant levels of RIPK1 mRNA co-immunoprecipitated with FMRP as compared to an IgG isotype control.

To confirm the association of FMRP with components of the necroptosis pathway, cells were left untreated or transfected with either FMRP antisense oligonucleotide (final concentration 0.5 nM and 100 nM) or FMRP sense oligonucleotide (final concentration 100 nM); incubated with a pMLKL inhibitor (necrosulfonamide, NSA; final concentration 1 μM)(Calbiochem); or a pRIPK1 inhibitor (necrostatinl, NEC1; final concentration 10 μM)(Cayman Chemical, Ann Arbor, Mich. USA). After 24 hours cells were collected and analyzed by Western blot, or stained with FITC-Annexin V and PI for flow cytometry analysis, as previously described in Examples 1-3.

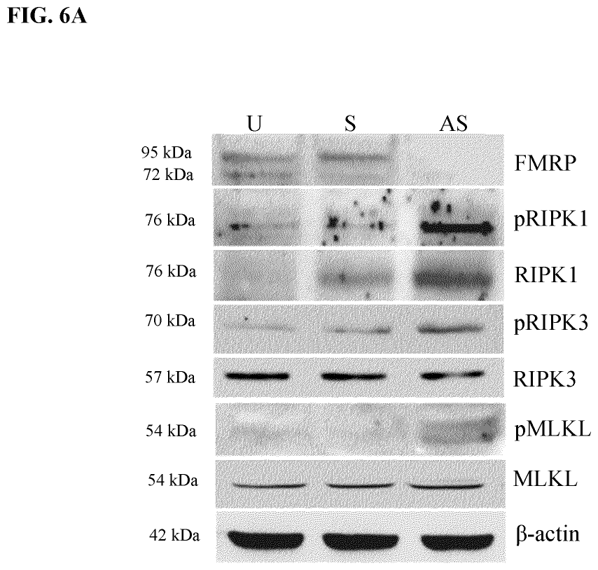

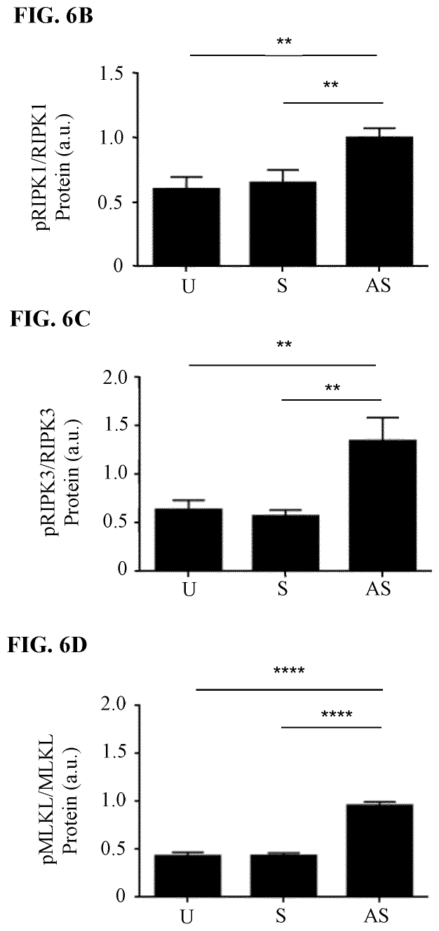

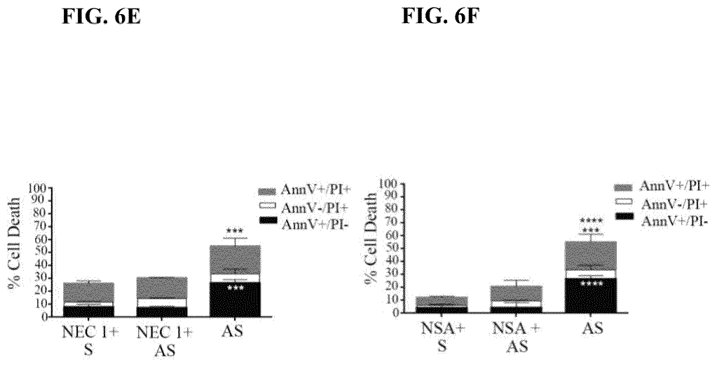

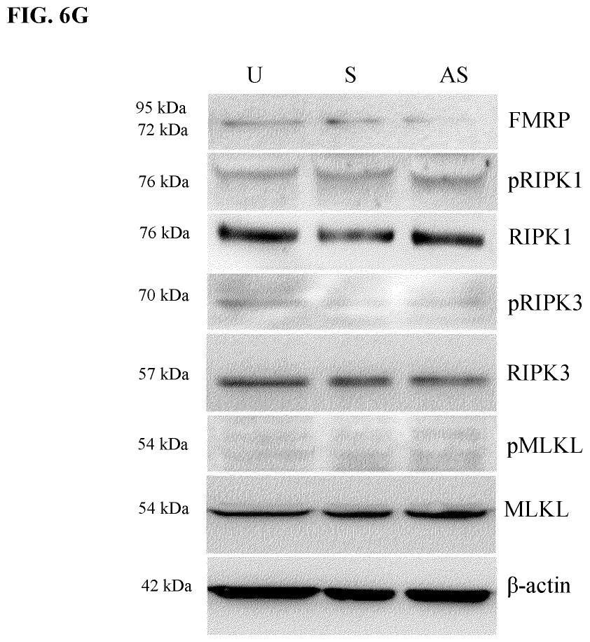

As shown in FIGS. 6A-6D, treatment with FMRP antisense oligonucleotide increased the total protein expression levels of RIPK1 in human CRC cell lines. FMRP anti sense oligonucleotide treatment additionally resulted in increased phosphorylation of RIPK1, RIPK3, and MLKL. However, as seen in FIG. 6G, similar increases in phosphorylation were not observed in HCEC-1ct cells treated with FMRP antisense oligonucleotide.

As shown in FIGS. 6E and 6F, human CRC cell lines incubated with the RIPK1-specific inhibitor, necrostatin 1 (NEC1), and the MLKL-specific inhibitor, necrosulfonamide (NSA), were protected from FMRP antisense oligonucleotide-induced cell death. Collectively, these results suggest that FMRP abrogates necroptosis in CRC cells by inhibiting the RIPK1 signaling cascade, thereby suppressing necroptosis.

### Example 5: FMRP Expression is Regulated by CREB

Several intracellular protein kinases, e.g., mTOR and MAPK, and transcription factors, e.g., CREB have been reported to positively regulate FMRP expression. This example demonstrates that FMRP expression is positively regulated by the CREB transcription factor.

CREB mRNA Expression and Protein Expression

To asses if CREB regulates FMRP expression, CRC cell lines were transfected with either CREB antisense (ASc) (5′-GCATCTCCACTCTGCTGGTT-3′) (SEQ ID NO: 16) or CREB sense (Ss) (5′-AACCAGCAGAGTGGAGATGC-3′)(SEQ ID NO: 17)(final concentration 200 nM) for 24 or 48 hours. mRNA and total protein lysates were prepared and analyzed by RT-PCR and Western blot, respectively, as previously described in Examples 1 to 3.

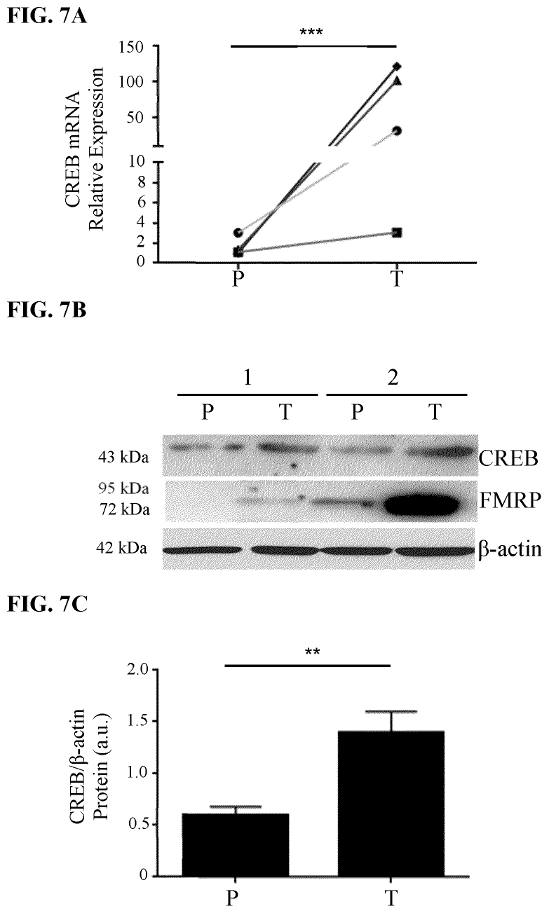

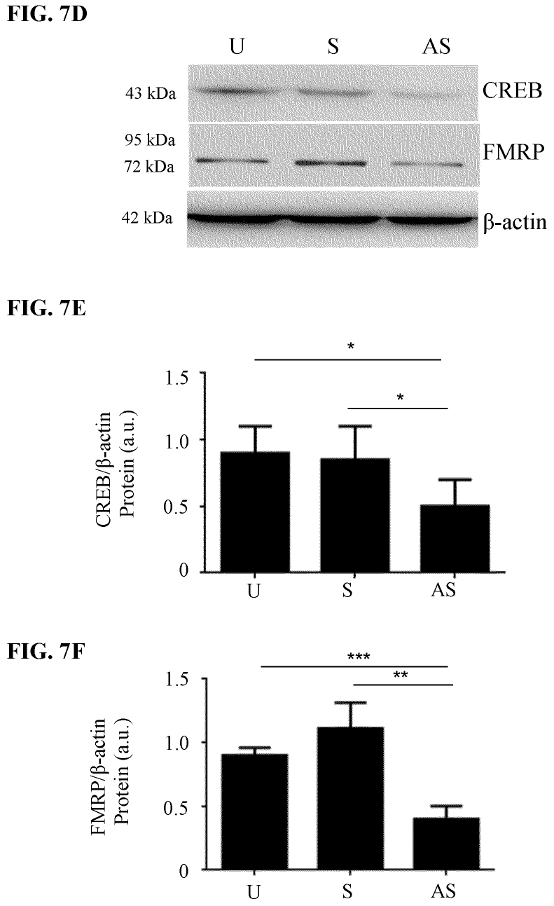

As shown in FIG. 7A, human CRC tumors (T) had significantly higher levels of CREB mRNA transcripts as compared to levels in peritumor samples (P). Similarly, as shown in FIGS. 7B and 7C, CREB protein expression is significantly higher in human CRC tumors (T) as compared to peritumor tissue samples (P).

To investigate whether CREB regulates FMRP expression in CRC cell lines, CREB expression was inhibited using a specific AS oligonucleotide. As shown in FIGS. 7D-7F, human CRC cell lines treated with CREB antisense oligonucleotide had reduced expression of CREB protein as compared to untreated cells or cells treated with sense oligonucleotide control. Furthermore, treatment with CREB antisense oligonucleotide resulted in a concomitant reduction in FMRP protein expression. These findings suggest that in human CRC, CREB positively regulates FMRP expression.

### Example 6: FMRP Levels Affect CRC Migration and Invasion

To investigate the role of FMRP in CRC migration and invasion, in vitro models of wound closure and cell invasion were used. This example describes that FMRP regulates proteins involved in cell migration and invasion including: E-cadherin, β-catenin, and mutated in colorectal cancer (MCC), a tumor suppressor whose gene is silenced by promoter methylation in colorectal cancer and particularly in patients with increased lymph node metastases.

### FMRP Levels Affect CRC Cell Migration and Invasion by E-Cadherin and Fi-Catenin Regulation

To investigate the role of FMRP in tumor cell migration and invasion, HCT-116 cells were seeded in each side of an Ibidi® culture insert and grown to confluence then left untreated (U) or transfected with sense FMRP oligonucleotide (S) (final concentration 0.5 nM) or antisense FMRP oligonucleotide (AS) (final concentration 0.5 nM). Additionally, HCT-116 cells were seeded in Transwell® inserts precoated with Matrigel®, and either left untreated (U) or transfected with sense oligonucleotide (S) (final concentration 100 nM) or antisense FMRP oligonucleotide (AS) (final concentration 0.5 nM) for 48 hours.

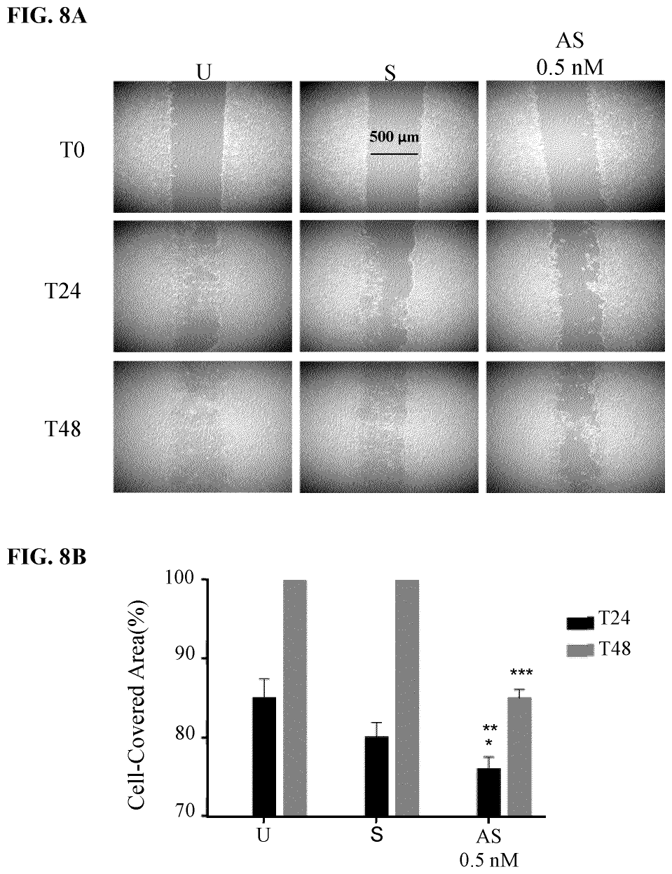

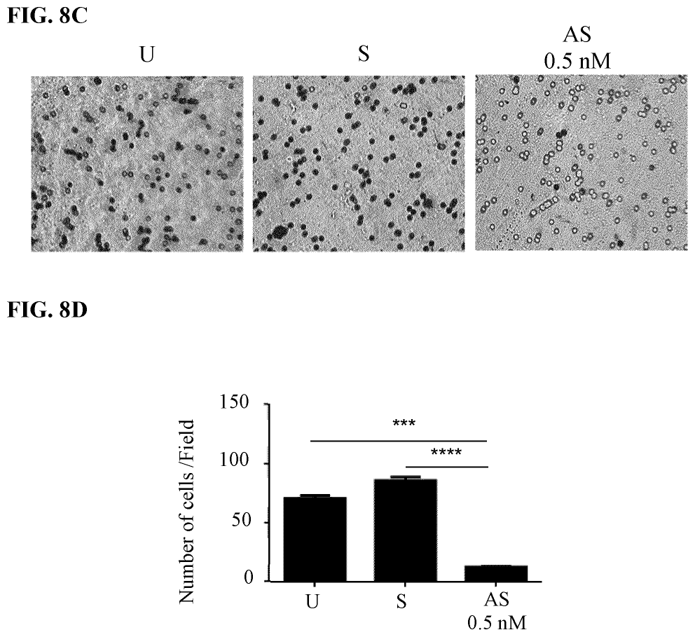

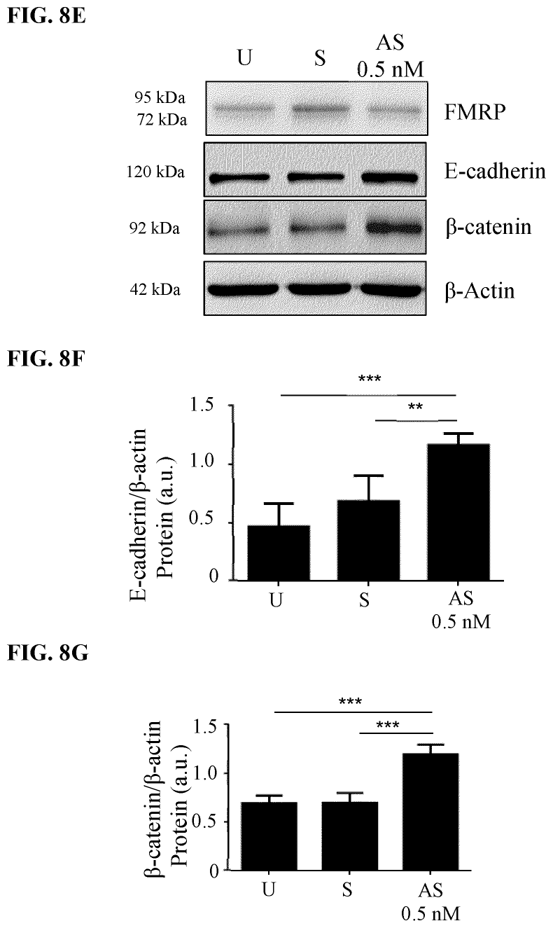

As shown in FIGS. 8A and 8B, transfection of HCT-116 cells with antisense FMRP oligonucleotide significantly reduced the percentage of cells covering a pseudo-“wound” as compared to untreated cells or cells transfected with sense oligonucleotide. The graph indicates the mean percentage of cell-covered area ±S.D. of 3 separate experiments.

Similarly, as shown in FIGS. 8C and 8D, HCT-116 cells transfected with antisense FMRP oligonucleotide exhibited significantly reduced migration across a Matrigel-coated Transwell® insert as compared to untreated cells or cells transfected with sense oligonucleotide. The graph indicates the mean number of migrating cells ±S.D. of 3 independent experiments.

To determine whether FMRP regulates levels of E-cadherin and/or β-catenin, key proteins associated with cellular adhesion, cytoskeletal remodeling, and inhibition of tumor migration and invasion, cells were left untreated (U), or transfected with either FMRP sense (S) oligonucleotide (final concentration 0.5 nM) or FMRP antisense (AS) oligonucleotide (final concentration 0.5 nM) for 48 hours.

As shown in FIGS. 8E-8G, transfection of HCT-116 cells with antisense FMRP oligonucleotide significantly increased the expression of E-cadherin and β-catenin as compared to untreated cells or cells transfected with sense oligonucleotide. FIGS. 8F and 8G express values in arbitrary units (a.u.) and indicate the mean±S.D. of 3 separate experiments.

FMRP Down-Regulates MCC, a Protein that Regulates E-Cadherin and β-Catenin Expression

To investigate whether FMRP regulates MCC, a protein known to interact with the E-cadherin/β-catenin complex, HCT-116 cells were left untreated (U) or transfected with sense FMRP oligonucleotide (S) (final concentration 0.5 nM) or antisense FMRP oligonucleotide (AS) (final concentration 0.5 nM) for 48 hours.

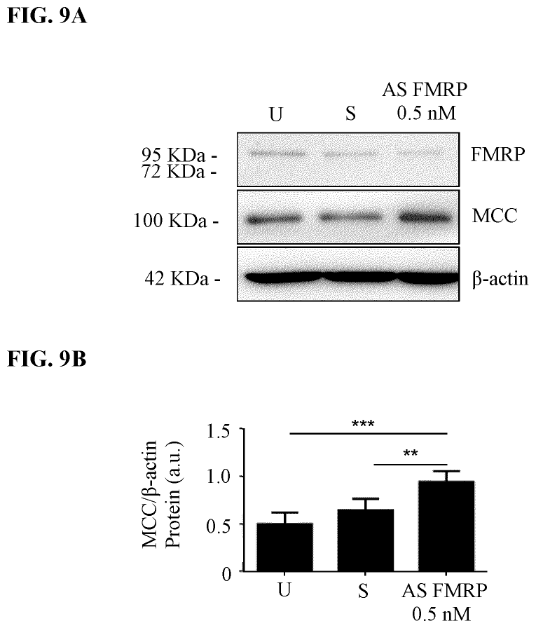

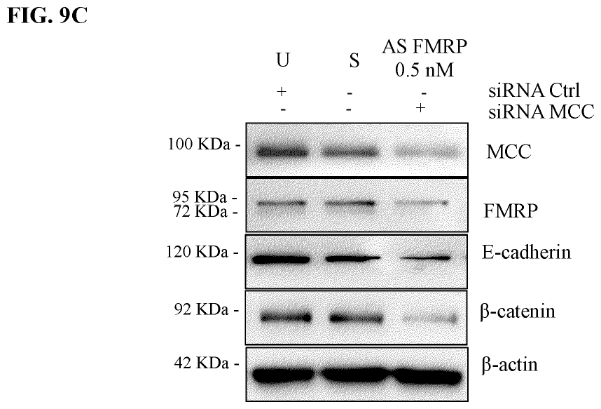

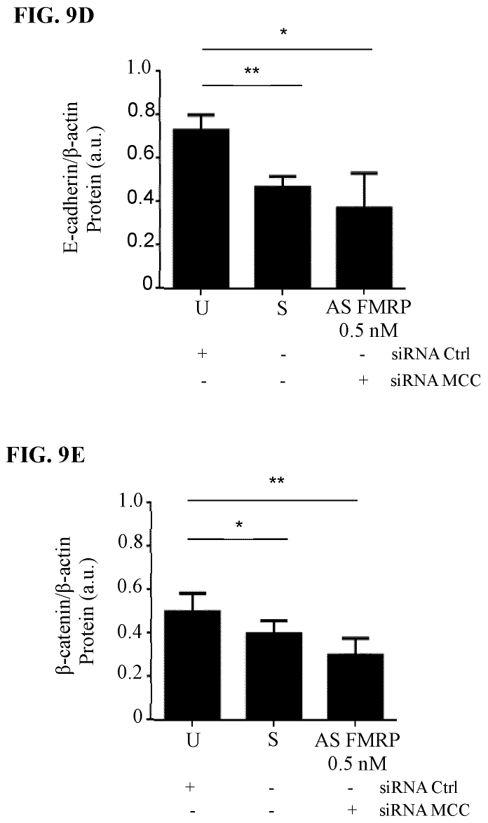

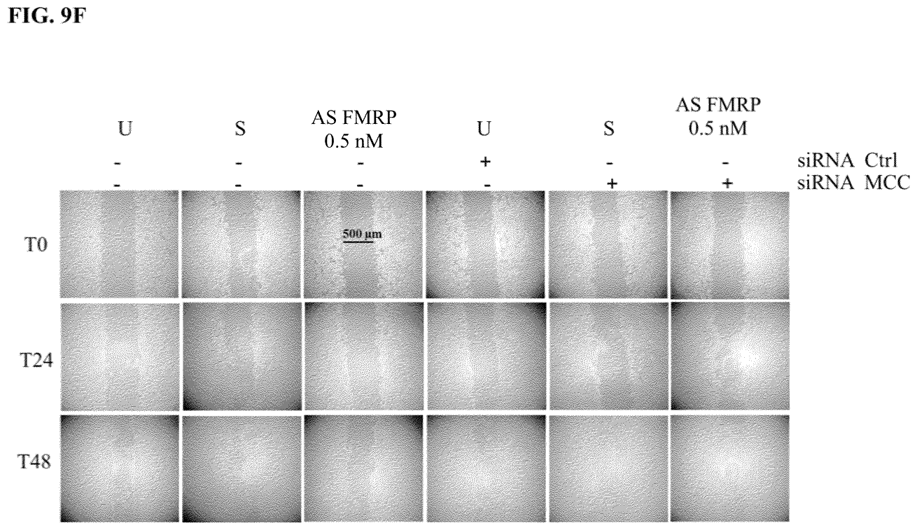

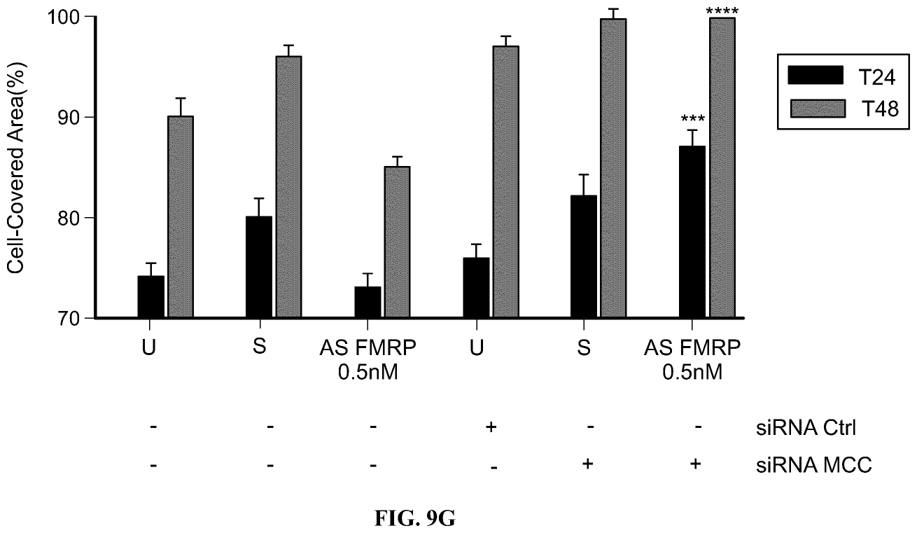

As shown in FIGS. 9A and 9B, HCT-116 cells transfected with antisense FMRP oligonucleotide exhibited significantly increased expression of MCC as compared to untreated cells or cells transfected with sense FMRP oligonucleotide.

Recent studies have described a novel tumor suppressor function of MCC in the regulation of E-cadherin/β-catenin complex-mediated cell-cell adhesion in colorectal cells. To investigate whether MCC mediates the observed increase of E-cadherin and β-catenin expression in HCT-116 cells transfected with antisense FMRP oligonucleotide (e.g., FIGS. 8E-8G), cells were left untreated (U) or transfected with sense FMRP oligonucleotide (final concentration 0.5 nM), or antisense FMRP oligonucleotide (final concentration 0.5 nM), and/or control siRNA (siRNA Ctrl) or siRNA specific for MCC (siRNA MCC) for 48 hours.

As shown in the representative Western blot (FIG. 9C) and corresponding quantitative analyses (FIGS. 9D and 9E), the presence of siRNA MCC abrogated the up-regulated expression of E-cadherin and β-catenin in cells transfected with antisense FMRP oligonucleotide alone.

To determine whether MCC regulation of the E-cadherin/β-catenin complex is also required for the observed inhibition of colorectal cell line migration and invasion (e.g., FIGS. 8A and 8B), HCT-116 cells were seeded in each side of an Ibidi® culture insert and grown to confluence then left untreated (U) or transfected with sense oligonucleotide (S) (final concentration 0.5 nM) or antisense oligonucleotide (AS) (final concentration 0.5 nM) and/or siRNA Ctrl or siRNA MCC.

As shown in FIGS. 9F and 9G, the presence of siRNA specific for MCC significantly reduced anti-sense FMRP oligonucleotide-mediated inhibition of colorectal cancer cell line migration and invasion. Together, these data suggest that FMRP controls cell-cell adhesion in colon cancer cells by regulating the MCC/E-cadherin/β-catenin complex.

### INCORPORATION BY REFERENCE

The entire disclosure of each of the patent documents and scientific articles cited herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A method of treating a bowel disease in a patient in need thereof comprising administering to the patient an effective amount of an antisense oligonucleotide that inhibits the expression of fragile X mental retardation protein (FMRP).

2. The method of claim 1, wherein the bowel disease is colorectal cancer.

3. The method of claim 1, wherein the bowel disease is an inflammatory bowel disease.

4. The method of claim 3, wherein the inflammatory bowel disease is Crohn's disease or ulcerative colitis.

5. A method of treating a solid tumor, tumor invasion, or tumor metastasis in a patient in need thereof comprising administering to the patient an effective amount of an antisense oligonucleotide that inhibits the expression of fragile X mental retardation protein (FMRP).

6. A method of preventing or ameliorating tumor invasion or tumor metastasis in a patient in need thereof comprising administering to the patient an effective amount of an antisense oligonucleotide that inhibits the expression of fragile X mental retardation protein (FMRP).

7. A method of preventing or ameliorating colorectal cancer tumor invasion or colorectal tumor metastasis in a patient in need thereof comprising administering to the patient an effective amount of an antisense oligonucleotide that inhibits the expression of fragile X mental retardation protein (FMRP).

8. The method of any one of claims 1 to 7, wherein the antisense oligonucleotide induces necroptosis.

9. The method of any one of claims 1 to 8, wherein the antisense oligonucleotide comprises a sequence selected from the group consisting of:
(SEQ ID NO: 1)
5′-CCACCACCAGCTCCTCCA-3′,
(SEQ ID NO: 2)
5′-CTTCCACCACCAGCTCCT-3′,
(SEQ ID NO: 3)
5′-TCCACCACCAGCTCCTCC-3′,
(SEQ ID NO: 4)
5′-CTTCCACCACCAGCTCC-3′,
and
(SEQ ID NO: 5)
5′-TCACCCTTTATCATCCTC-3′,
or a complement thereof.

10. The method of any one of claims 1 to 9, wherein the antisense oligonucleotide comprises a sequence selected from the group consisting of:
(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,
or a complement thereof.

11. The method of any one of claims 1 to 10, wherein the antisense oligonucleotide comprises one or more ribonucleotides.

12. The method of any one of claims 1 to 11, wherein the antisense oligonucleotide comprises one or more deoxyribonucleotides.

13. The method of any one of claims 1 to 12, wherein the antisense oligonucleotide comprises a mixture of ribonucleotides and deoxyribonucleotides.

14. The method of claims 1 to 13, wherein the antisense oligonucleotide comprises one or more modified nucleoside selected from the group consisting of 5-methylcytidine, 5-methyl-2′-deoxycytidine, deoxycytidine, 5-methyl-2′-deoxycytidine 5′-monophosphate, and 5-methyl-2′-deoxycytidine-5′-monophosphorothioate.

15. The method of claims 1 to 14, wherein the antisense oligonucleotide comprises one or more modified nucleoside selected from the group consisting of 2′-O-methylcytidine, 2′-O-methylguanosine, 2′-O-methylthymidine, 2′-O-methyluridine, and 2′-O-methyladenosine.

16. The method of claims 1 to 15, wherein the antisense oligonucleotide comprises one or more modified nucleotide selected from the group consisting of 5-methyl cytosine and 5-methylguanine.

17. The method of claims 1 to 16, wherein the antisense oligonucleotide comprises one or more modified nucleoside selected from 2′-O-(2-methoxyethyl) nucleosides, 2′-deoxy-2′-fluoro nucleosides, and 2′-fluoro-β-D-arabinonucleosides.

18. The method of claims 1 to 17, wherein the antisense oligonucleotide comprises one or more of the group selected from bridged nucleic acids, locked nucleic acids (LNA), constrained ethyl (cET) nucleic acids, tricyclo-DNAs (tcDNA), 2′-O,4′-C-ethylene linked nucleic acids (ENA), and peptide nucleic acids (PNA).

19. The method of any one of claims 1 to 18, wherein the antisense oligonucleotide comprises at least one internucleoside linkage selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

20. The method of any one of claims 1 to 19, wherein the antisense oligonucleotide comprises at least one phosphorothioate linkage.

21. The method of any one of claims 1 to 20, wherein all internucleoside linkages of the antisense oligonucleotide are phosphorothioate linkages.

22. The method of claim 19, wherein the anti sense oligonucleotide comprises at least one methylphosphonate linkage.

23. The method of any one of claims 1 to 22, wherein the antisense oligonucleotide is from 20 to 40 nucleotides in length.

24. The method of any one of claims 1 to 23, wherein the antisense oligonucleotide is from 20 to 24 nucleotides in length.

25. The method of any one of claims 1 to 24, wherein the antisense oligonucleotide consists of a sequence selected from the group consisting of:
(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,
or a complement thereof.

26. The method of claim 25, wherein the antisense oligonucleotide comprises at least one phosphorothioate linkage.

27. The method of claim 25 or 26, wherein all internucleoside linkages of the antisense oligonucleotide are phosphorothioate linkages.

28. The method of claim 25, wherein the anti sense oligonucleotide comprises at least one methylphosphonate linkage.

29. The method of claim 8, wherein the antisense oligonucleotide results in increased activation of one or more than one kinase selected from the group consisting of: receptor interacting protein kinase 1 (RIPK1), receptor interacting protein kinase 3 (RIPK3), and mixed lineage kinase domain-like protein (MLKL).

30. The method of any one of claims 1 to 29, wherein the oligonucleotide is administered to the patient enterally or parenterally.

31. The method of claim 30, wherein enteral administration is oral, sublingual, gastric, or rectal.

32. The method of claim 30, wherein parenteral administration is intravenous, intratumoral, intrajejunal, intraileal, intracolonic, or intrarectal.

33. The method according to any one of claims 1 to 32, wherein the patient is human.

34. An antisense oligonucleotide comprising a sequence selected from the group consisting of:
(SEQ ID NO: 1)
5′-CCACCACCAGCTCCTCCA-3′,
(SEQ ID NO: 2)
5′-CTTCCACCACCAGCTCCT-3′,
(SEQ ID NO: 3)
5′-TCCACCACCAGCTCCTCC-3′,
(SEQ ID NO: 4)
5′-CTTCCACCACCAGCTCC-3′,
and
(SEQ ID NO: 5)
5′-TCACCCTTTATCATCCTC-3′,
or a complement thereof.

35. The antisense oligonucleotide of claim 34, wherein the antisense oligonucleotide sequence comprises a sequence selected from the group consisting of:
(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,
or a complement thereof.

36. The antisense oligonucleotide of claim 34 or 35, wherein the antisense oligonucleotide comprises one or more ribonucleotides.

37. The antisense oligonucleotide of any one of claims 34 to 36, wherein the antisense oligonucleotide comprises one or more deoxyribonucleotides.

38. The antisense oligonucleotide of any one of claims 34 to 37, wherein the antisense oligonucleotide comprises a mixture of ribonucleotides and deoxyribonucleotides.

39. The antisense oligonucleotide of any one of claims 34 to 38, wherein the antisense oligonucleotide comprises one or more modified nucleoside selected from the group consisting of 5-methylcytidine, 5-methyl-2′-deoxycytidine, deoxycytidine, 5-methyl-2′-deoxycytidine 5′-monophosphate, and 5-methyl-2′-deoxycytidine-5′-monophosphorothioate.

40. The antisense oligonucleotide of any one of claims 34 to 39, wherein the antisense oligonucleotide comprises one or more modified nucleoside selected from the group consisting of 2′-O-methylcytidine, 2′-O-methylguanosine, 2′-O-methylthymidine, 2′-O-methyluridine, and 2′-O-methyladenosine.

41. The antisense oligonucleotide of any one of claims 34 to 40, wherein the antisense oligonucleotide comprises one or more modified nucleotide selected from the group consisting of 5-methyl cytosine and 5-methylguanine.

42. The antisense oligonucleotide of any one of claims 34 to 41, wherein the antisense oligonucleotide comprises one or more modified nucleoside selected from 2′-O-(2-methoxyethyl) nucleosides, 2′-deoxy-2′-fluoro nucleosides, and 2′-fluoro-β-D-arabinonucleosides.

43. The antisense oligonucleotide of any one of claims 34 to 42, wherein the antisense oligonucleotide comprises one or more of the group selected from bridged nucleic acids, locked nucleic acids (LNA), constrained ethyl (cET) nucleic acids, tricyclo-DNAs (tcDNA), 2′-O,4′-C-ethylene linked nucleic acids (ENA), and peptide nucleic acids (PNA).

44. The antisense oligonucleotide of any one of claims 34 to 43, wherein the antisense oligonucleotide comprises at least one internucleoside linkage selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

45. The antisense oligonucleotide of any one of claims 34 to 44, wherein the antisense oligonucleotide comprises at least one phosphorothioate linkage.

46. The antisense oligonucleotide of any one of claims 34 to 45, wherein all internucleoside linkages of the antisense oligonucleotide are phosphorothioate linkages.

47. The antisense oligonucleotide sequence of any one of claims 34 to 44, wherein the antisense oligonucleotide comprises at least one methylphosphonate linkage.

48. The antisense oligonucleotide of any one of claims 34 to 47, wherein the antisense oligonucleotide is from 20 to 40 nucleotides in length.

49. The antisense oligonucleotide of any one of claims 34 to 48, wherein the antisense oligonucleotide is from 20 to 24 nucleotides in length.

50. The antisense oligonucleotide of any one of claims 34 to 49, wherein the antisense oligonucleotide sequence consists of a sequence selected from the group consisting of:
(SEQ ID NO: 6)
5′-TCCACCACCAGCTCCTCCAT-3′,
(SEQ ID NO: 7)
5′-ACTTCCACCACCAGCTCCTC-3′,
(SEQ ID NO: 8)
5′-TTCCACCACCAGCTCCTCCA-3′,
(SEQ ID NO: 9)
5′-ACTTCCACCACCAGCTCCT-3′,
and
(SEQ ID NO: 10)
5′-CTCACCCTTTATCATCCTCA-3′,
or a complement thereof.

51. The antisense oligonucleotide of claim 50, wherein the antisense oligonucleotide comprises at least one phosphorothioate linkage.

52. The antisense oligonucleotide of claim 50 or 51, wherein all internucleoside linkages of the antisense oligonucleotide are phosphorothioate linkages.

53. The antisense oligonucleotide of claim 50, wherein the antisense oligonucleotide comprises at least one methylphosphonate linkage.

54. The antisense oligonucleotide of claim 34 or 35, wherein the antisense oligonucleotide is an FMRP siRNA, or a pharmaceutically acceptable salt thereof.

55. The FMRP siRNA of claim 54, wherein the FMRP siRNA comprises at least one internucleoside linkage selected from the group consisting of a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, a phosphoromorpholidate linkage, a phosphoropiperazidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

56. The FMRP siRNA of claim 54 or 55, wherein the FMRP siRNA comprises at least one phosphorothioate linkage.

57. The FMRP siRNA of any one of claims 54 to 56, wherein all nucleoside linkages of the FMRP siRNA are phosphorothioate linkages.

58. The FMRP siRNA of any one of claims 54 to 57, wherein at least one cytidine of the FMRP siRNA is replaced with 5-methylcytidine.

59. The FMRP siRNA of any one of claims 54 to 58, wherein the FMRP siRNA is 20, 21, 22, 23, 24, 25, 26, 27, 28, 29, 30, 31, 32, 33, 34, 35, 36, 37, 38, 39, 40, 20 to 40, or 20 to 24 nucleotides in length.

60. The FMRP siRNA of any one of claims 54 to 59, wherein the FMRP siRNA is from 20 to 40 nucleotides in length.

61. The FMRP siRNA of any one of claims 54 to 60, wherein the FMRP siRNA is from 20 to 24 nucleotides in length.

62. A pharmaceutically acceptable composition comprising an antisense oligonucleotide according to any one of claims 32 to 54, or an FMRP siRNA of any one of claims 55 to 61; and a pharmaceutically acceptable carrier.

63. Use of an antisense oligonucleotide according to any one of claims 32 to 54, or an FMRP siRNA of any one of claims 55 to 61, in the manufacture of a medicament for the treatment of a bowel disease.

64. The use of claim 63, wherein the bowel disease is colorectal cancer.

65. The use of claim 63, wherein the bowel disease is an inflammatory bowel disease.

66. The use of claim 65, wherein the inflammatory bowel disease is Crohn's disease or ulcerative colitis.

67. The use of any one of claims 63 to 66, wherein the medicament is administered to the patient enterally or parenterally.

68. The use of claim 67, wherein enteral administration is oral, sublingual, gastric, or rectal.

69. The use of claim 67, wherein parenteral administration is intravenous, intratumoral, intrajejunal, intraileal, intracolonic, or intrarectal.

70. The use of any one of claims 63 to 69, wherein the medicament is for treatment of a bowel disease in a human.

71. Use of an antisense oligonucleotide according to any one of claims 32 to 54, or an FMRP siRNA of any one of claims 55 to 61, in the manufacture of a medicament for the treatment of a solid tumor, tumor invasion, or tumor metastasis.


SEQUENCE LISTING (22 sequences)

SEQ ID NO: 1 - DNA, 18 bp, Artificial Sequence
  Note= Description of Artificial Sequence Antisense Oligonucleotide
ccaccaccagctcctcca

SEQ ID NO: 2 - DNA, 18 bp, Artificial Sequence
  Note= Description of Artificial Sequence Antisense Oligonucleotide
cttccaccaccagctcct

SEQ ID NO: 3 - DNA, 18 bp, Artificial Sequence
  Note= Description of Artificial Sequence Antisense Oligonucleotide
tccaccaccagctcctcc

SEQ ID NO: 4 - DNA, 17 bp, Artificial Sequence
  Note= Description of Artificial Sequence Antisense Oligonucleotide
cttccaccaccagctcc

SEQ ID NO: 5 - DNA, 18 bp, Artificial Sequence
  Note= Description of Artificial Sequence Antisense Oligonucleotide
tcaccctttatcatcctc

SEQ ID NO: 6 - DNA, 20 bp, Artificial Sequence
  Note= Description of Artificial Sequence Antisense Oligonucleotide
tccaccaccagctcctccat

SEQ ID NO: 7 - DNA, 20 bp, Artificial Sequence
  Note= Description of Artificial Sequence Antisense Oligonucleotide
acttccaccaccagctcctc

SEQ ID NO: 8 - DNA, 20 bp, Artificial Sequence
  Note= Description of Artificial Sequence Antisense Oligonucleotide
ttccaccaccagctcctcca

SEQ ID NO: 9 - DNA, 19 bp, Artificial Sequence
  Note= Description of Artificial Sequence Antisense Oligonucleotide
acttccaccaccagctcct

SEQ ID NO: 10 - DNA, 20 bp, Artificial Sequence
  Note= Description of Artificial Sequence Antisense Oligonucleotide
ctcaccctttatcatcctca

SEQ ID NO: 11 - DNA, 22 bp, Artificial Sequence
  Note= Description of Artificial Sequence Synthetic primer (human FMRP)
gttgagcggccgagtttgtcag

SEQ ID NO: 12 - DNA, 24 bp, Artificial Sequence
  Note= Description of Artificial Sequence Synthetic primer (human FMRP)
cccactgggagaggattatttggg

SEQ ID NO: 13 - DNA, 27 bp, Artificial Sequence
  Note= Description of Artificial Sequence Synthetic primer (human and mouse b-actin)
aagatgacccagatcatgtttgagacc

SEQ ID NO: 14 - DNA, 20 bp, Artificial Sequence
  Note= Description of Artificial Sequence Synthetic primer (human and mouse b-actin)
agccagtccagacgcaggat

SEQ ID NO: 15 - DNA, 20 bp, Artificial Sequence
  Note= Description of Artificial Sequence Antisense Oligonucleotide
atggaggagctggtggtgga

SEQ ID NO: 16 - DNA, 20 bp, Artificial Sequence
  Note= Description of Artificial Sequence Antisense Oligonucleotide
gcatctccactctgctggtt

SEQ ID NO: 17 - DNA, 20 bp, Artificial Sequence
  Note= Description of Artificial Sequence Sense Oligonucleotide
aaccagcagagtggagatgc

SEQ ID NO: 18 - DNA, 4140 bp, Homo sapiens
acttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaatt

SEQ ID NO: 19 - DNA, 4348 bp, Homo sapiens
acttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaatt

SEQ ID NO: 20 - DNA, 4107 bp, Homo sapiens
ctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaa

SEQ ID NO: 21 - DNA, 4303 bp, Homo sapiens
ctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagaggggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaa

SEQ ID NO: 22 - DNA, 4441 bp, Homo sapiens
ctcagtcaggcgctcagctccgtttcggtttcacttccggtggagggccgcctctgagcgggcggcgggccgacggcgagcgcgggcggcggcggtgacggaggcgccgctgccagggggcgtgcggcagcgcggcggcggcggcggcggcggcggcggcggaggcggcggcggcggcggcggcggcggcggctgggcctcgagcgcccgcagcccacctctcgggggcgggctcccggcgctagcagggctgaagagaagatggaggagctggtggtggaagtgcggggctccaatggcgctttctacaaggcatttgtaaaggatgttcatgaagattcaataacagttgcatttgaaaacaactggcagcctgataggcagattccatttcatgatgtcagattcccacctcctgtaggttataataaagatataaatgaaagtgatgaagttgaggtgtattccagagcaaatgaaaaagagccttgctgttggtggttagctaaagtgaggatgataaagggtgagttttatgtgatagaatatgcagcatgtgatgcaacttacaatgaaattgtcacaattgaacgtctaagatctgttaatcccaacaaacctgccacaaaagatactttccataagatcaagctggatgtgccagaagacttacggcaaatgtgtgccaaagaggcggcacataaggattttaaaaaggcagttggtgccttttctgtaacttatgatccagaaaattatcagcttgtcattttgtccatcaatgaagtcacctcaaagcgagcacatatgctgattgacatgcactttcggagtctgcgcactaagttgtctctgataatgagaaatgaagaagctagtaagcagctggagagttcaaggcagcttgcctcgagatttcatgaacagtttatcgtaagagaagatctgatgggtctagctattggtactcatggtgctaatattcagcaagctagaaaagtacctggggtcactgctattgatctagatgaagatacctgcacatttcatatttatggagaggatcaggatgcagtgaaaaaagctagaagctttctcgaatttgctgaagatgtaatacaagttccaaggaacttagtaggcaaagtaataggaaaaaatggaaagctgattcaggagattgtggacaagtcaggagttgtgagggtgaggattgaggctgaaaatgagaaaaatgttccacaagaagaggaaattatgccaccaaattcccttccttccaataattcaagggttggacctaatgccccagaagaaaaaaaacatttagatataaaggaaaacagcacccatttttctcaacctaacagtacaaaagtccagagggtgttagtggcttcatcagttgtagcaggggaatcccagaaacctgaactcaaggcttggcagggtatggtaccatttgtttttgtgggaacaaaggacagcatcgctaatgccactgttcttttggattatcacctgaactatttaaaggaagtagaccagttgcgtttggagagattacaaattgatgagcagttgcgacagattggagctagttctagaccaccaccaaatcgtacagataaggaaaaaagctatgtgactgatgatggtcaaggaatgggtcgaggtagtagaccttacagaaatagggggcacggcagacgcggtcctggatatacttcaggaactaattctgaagcatcaaatgcttctgaaacagaatctgaccacagagacgaactcagtgattggtcattagctccaacagaggaagagagggagagcttcctgcgcagaggagacggacggcggcgtggagggggaggaagaggacaaggaggaagaggacgtggaggaggcttcaaaggaaacgacgatcactcccgaacagataatcgtccacgtaatccaagagaggctaaaggaagaacaacagatggatcccttcagatcagagttgactgcaataatgaaaggagtgtccacactaaaacattacagaatacctccagtgaaggtagtcggctgcgcacgggtaaagatcgtaaccagaagaaagagaagccagacagcgtggatggtcagcaaccactcgtgaatggagtaccctaaactgcataattctgaagttatatttcctataccatttccgtaattcttattccatattagaaaactttgttaggccaaagacaaatagtaggcaagatggcacagggcatgaaatgaacacaaattatgctaagaattttttattttttggtattggccataagcaacaattttcagatttgcacaaaaagataccttaaaatttgaaacattgcttttaaaactacttagcacttcagggcagattttagttttattttctaaagtactgagcagtgatattctttgttaatttggaccattttcctgcattgggtgatcattcaccagtacattctcagtttttcttaatatatagcatttatggtaatcatattagacttctgttttcaatctcgtatagaagtcttcatgaaatgctatgtcatttcatgtcctgtgtcagtttatgttttggtccacttttccagtattttagtggaccctgaaatgtgtgtgatgtgacatttgtcattttcattagcaaaaaaagttgtatgatctgtgccttttttatatcttggcaggtaggaatattatatttggatgcagagttcagggaagataagttggaaacactaaatgttaaagatgtagcaaaccctgtcaaacattagtactttatagaagaatgcatgctttccatatttttttccttacataaacatcaggttaggcagtataaagaataggacttgtttttgtttttgttttgttgcactgaagtttgataaatagtgttattgagagagatgtgtaatttttctgtatagacaggagaagaaagaactatcttcatctgagagaggctaaaatgttttcagctaggaacaaatcttcctggtcgaaagttagtaggatatgcctgctctttggcctgatgaccaattttaacttagagctttttttttttaattttgtctgccccaagttttgtgaaatttttcatattttaatttcaagcttattttggagagataggaaggtcatttccatgtatgcataataatcctgcaaagtacaggtactttgtctaagaaacattggaagcaggttaaatgttttgtaaactttgaaatatatggtctaatgtttaagcagaattggaaaagactaagatcggttaacaaataacaacttttttttctttttttcttttgttttttgaagtgttggggtttggttttgttttttgagtcttttttttttaagtgaaatttattgaggaaaaatatgtgaaggaccttcactctaagatgttatatttttcttaaaaagtaactcctagtaggggtaccactgaatctgtacagagccgtaaaaactgaagttctgcctctgatgtattttgtgagtttgtttctttgaattttcattttacagttacttttccttgcatacaaacaagcatataaaatggcaacaaactgcacatgatttcacaaatattaaaaagtcttttaaaaagtattgccaaacattaatgttgatttctagttatttattctgggaatgtatagtatttgaaaacagaaattggtaccttgcacacatcatctgtaagctgtttggttttaaaatactgtagataattaaccaaggtagaatgaccttgtaatgtaactgctcttgggcaatattctctgtacatattagcgacaacagattggattttatgttgacatttgtttggttatagtgcaatatattttgtatgcaagcagtttcaataaagtttgatcttcctctgctaaattgatgttgatgcaatccttacaaatgattgcttttaaaattttaagctaggaaaagaaatctatagaaagtgttctgttacaaaatgtaactgttaccattggaaatttcacgtcataggaagttagcctttatctaccaactttcaagaacttgtttaataaagcgaaaaactcaaccaaatggtacaaaaccacagtgtaccattaaaatatgcactaagtctcttttttacaaaggctgtattcagcaaggcgctaacttgcttaaatgtgaattactaacttctaaaactgtactttgattcacatgttttcaaatggagttggagttcattcatattacaatatttgtgtgctaaacgtgtatgtttttcagttcaaagtcatgatgtttttaaaatcttattaaagtttcaaaaatctgaagattgtttatctagatgtaaatttttattaaaaagttgcacttatgaaaaagcaaaaaa